(19) United States
(12) Patent Application Publication
(10) Pub. No.: US 2011/0023149 A1
(43) Pub. Date: Jan. 27, 2011

(54) GENOMIC EDITING OF GENES INVOLVED IN TUMOR SUPPRESSION IN ANIMALS

(75) Inventors: Edward Weinstein, St. Louis, MO (US); Xiaoxia Cui, St. Louis, MO (US); Phil Simmons, St. Louis, MO (US)

Correspondence Address: POLSINELLI SHUGHART PC, 700 W. 47TH STREET, SUITE 1000, KANSAS CITY, MO, 64112-1802, US

(73) Assignee: SIGMA-ALDRICH CO., St. Louis, MO (US)

(21) Appl. No.: 12/842,978

(22) Filed: Jul. 23, 2010

Related U.S. Application Data
(63) Continuation-in-part of application No. 12/592,852, filed on Dec. 3, 2009.
(60) Provisional application No. 61/343,287, filed on Apr. 26, 2010.
(60) Provisional application No. 61/323,702, filed on Apr. 13, 2010.
(60) Provisional application No. 61/323,719, filed on Apr. 13, 2010.
(60) Provisional application No. 61/323,698, filed on Apr. 13, 2010.
(60) Provisional application No. 61/309,729, filed on Mar. 2, 2010.
(60) Provisional application No. 61/308,089, filed on Feb. 25, 2010.
(60) Provisional application No. 61/336,000, filed on Jan. 14, 2010.
(60) Provisional application No. 61/263,904, filed on Nov. 24, 2009.
(60) Provisional application No. 61/263,696, filed on Nov. 23, 2009.
(60) Provisional application No. 61/245,877, filed on Sep. 25, 2009.
(60) Provisional application No. 61/232,620, filed on Aug. 10, 2009.
(60) Provisional application No. 61/228,419, filed on Jul. 24, 2009.
(60) Provisional application No. 61/200,985, filed on Dec. 4, 2008.
(60) Provisional application No. 61/205,970, filed on Jan. 26, 2009.

(51) Int. Cl. A61K 49/00; A01K 67/027; C12N 5/10; C12N 9/16; C07H 21/04; A61P 43/00

(52) U.S. Cl. 800/3; 800/13; 800/14; 800/15; 800/17; 800/18; 800/16; 435/325; 435/350; 435/351; 435/352; 435/363; 435/353; 435/196; 536/23.1

(57) ABSTRACT

The present invention provides genetically modified animals and cells comprising edited chromosomal sequences involved in tumor suppression. In particular, the animals or cells are generated using a zinc finger nuclease-mediated editing process. The invention also provides zinc finger nucleases that target chromosomal sequence involved in tumor suppression and the nucleic acids encoding the zinc finger nucleases. Also provided are methods of assessing the effects of agents in genetically modified animals and cells comprising edited chromosomal sequences involved in tumor suppression.

### CROSS-REFERENCE TO RELATED APPLICATIONS

This application claims the priority of U.S. provisional application No. 61/343,287, filed Apr. 26, 2010, U.S. provisional application No. 61/323,702, filed Apr. 13, 2010, U.S. provisional application No. 61/323,719, filed Apr. 13, 2010, U.S. provisional application No. 61/323,698, filed Apr. 13, 2010, U.S. provisional application No. 61/309,729, filed Mar. 2, 2010, U.S. provisional application No. 61/308,089, filed Feb. 25, 2010, U.S. provisional application No. 61/336,000, filed Jan. 14, 2010, U.S. provisional application No. 61/263,904, filed Nov. 24, 2009, U.S. provisional application No. 61/263,696, filed Nov. 23, 2009, U.S. provisional application No. 61/245,877, filed Sep. 25, 2009, U.S. provisional application No. 61/232,620, filed Aug. 10, 2009, U.S. provisional application No. 61/228,419, filed Jul. 24, 2009, and is a continuation in part of U.S. non-provisional application Ser. No. 12/592,852, filed Dec. 3, 2009, which claims priority to U.S. provisional 61/200,985, filed Dec. 4, 2008 and U.S. provisional application 61/205,970, filed Jan. 26, 2009, all of which are hereby incorporated by reference in their entirety.

### FIELD OF THE INVENTION

The invention generally relates to genetically modified animals or cells comprising at least one edited chromosomal sequence encoding a protein involved in tumor suppression. In particular, the invention relates to the use of a zinc finger nuclease-mediated process to edit chromosomal sequences encoding proteins involved in tumor suppression.

### BACKGROUND OF THE INVENTION

Tumor suppressors help regulate cell division and cell death. Mutations in tumor suppressor genes can lead to abnormal cell division and uncontrolled cell division, which is responsible for many types of cancer. This influx of cells in the body can grow into tissues, leading to specific cancers associated with various organs. Additionally, uncontrolled cell growth is responsible for blood cancer, such as leukemia. Half of all men and one-third of all women in the United States will develop cancer during their lifetimes. While research is being done to develop with new treatments for cancer, new treatment strategies and therapies are needed.

The vast majority of drugs (approximately 91%) fail to successfully proceed through the three phases of drug testing in humans. Several drugs fail do so because of unforeseen toxicology in human patients, despite the fact that all of these drugs had been tested in animal models and were found to be safe. This is because toxicology testing is performed in animals, and animal proteins differ from the orthologous proteins in humans.

What is needed are animals that are mutated for the genes involved in tumor suppression processes, including knockouts, multiple mutant lines (double knockouts, triple knockouts, etc.) and/or over-expression of alleles that either cause disease or are associated with disease in humans, as well as “humanized” animals that express or over-express human homologues of relevant genes in animals. Such animals could serve as research tools to develop and/or test new treatments for cancer.

### SUMMARY OF THE INVENTION

One aspect of the present disclosure encompasses a genetically modified animal comprising at least one edited chromosomal sequence encoding a sequence involved in tumor suppression.

A further aspect provides a non-human embryo comprising at least one RNA molecule encoding a zinc finger nuclease that recognizes a chromosomal sequence encoding a protein involved in tumor suppression, and, optionally, at least one donor polynucleotide comprising a sequence encoding a protein involved in tumor suppression.

Another aspect provides a genetically modified cell comprising at least one edited chromosomal sequence encoding a protein involved in tumor suppression.

An alternate aspect provides a zinc finger nuclease comprising (a) a zinc finger DNA binding domain that binds a sequence having at least about 80% sequence identity to a sequence chosen from SEQ ID NOs:3, 4, 5, 6, 7, and 8; and (b) a cleavage domain.

A further aspect provides a nucleic acid sequence recognized by a zinc finger nuclease. The nucleic acid sequence has at least about 80% sequence identity to a sequence chosen from SEQ ID NOs:3, 4, 5, 6, 7, and 8.

Yet another aspect encompasses a method for assessing the therapeutic effect and/or toxicity of an agent. The method comprises (a) contacting a first genetically modified animal comprising at least one edited chromosomal sequence encoding a protein involved in tumor suppressor with the agent; (b) measuring a therapeutic and/or toxicity response in the first animal; and (c) comparing the response in (b) to results obtained from a second genetically modified animal comprising the same edited chromosomal sequence encoding a protein involved in tumor suppression, wherein the second animal is not contacted with the agent.

Other aspects and features of the disclosure are described more thoroughly below.

### REFERENCE TO COLOR FIGURES

The application file contains at least one figure executed in color. Copies of this patent application publication with color figures will be provided by the Office upon request and payment of the necessary fee.

### BRIEF DESCRIPTION OF THE FIGURES

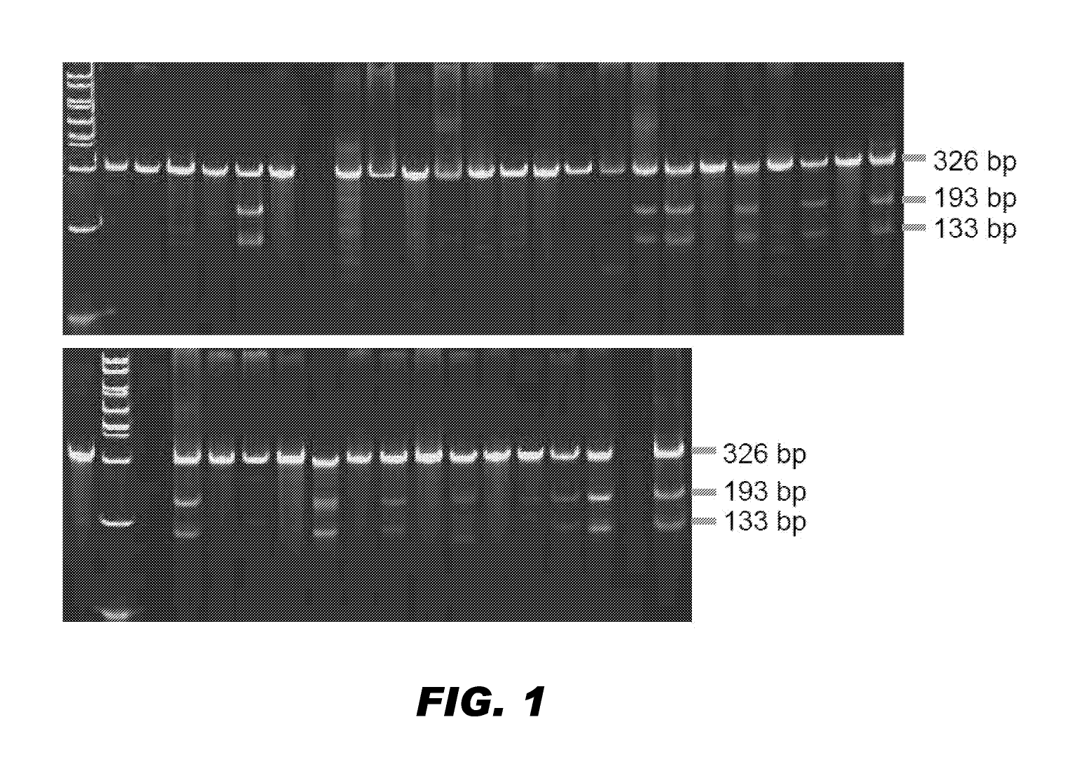

FIG. 1 illustrates editing of the p53 locus in rats. Presented is a Cel-1 assay in which the presence of cleavage products indicated editing of the p53 gene.

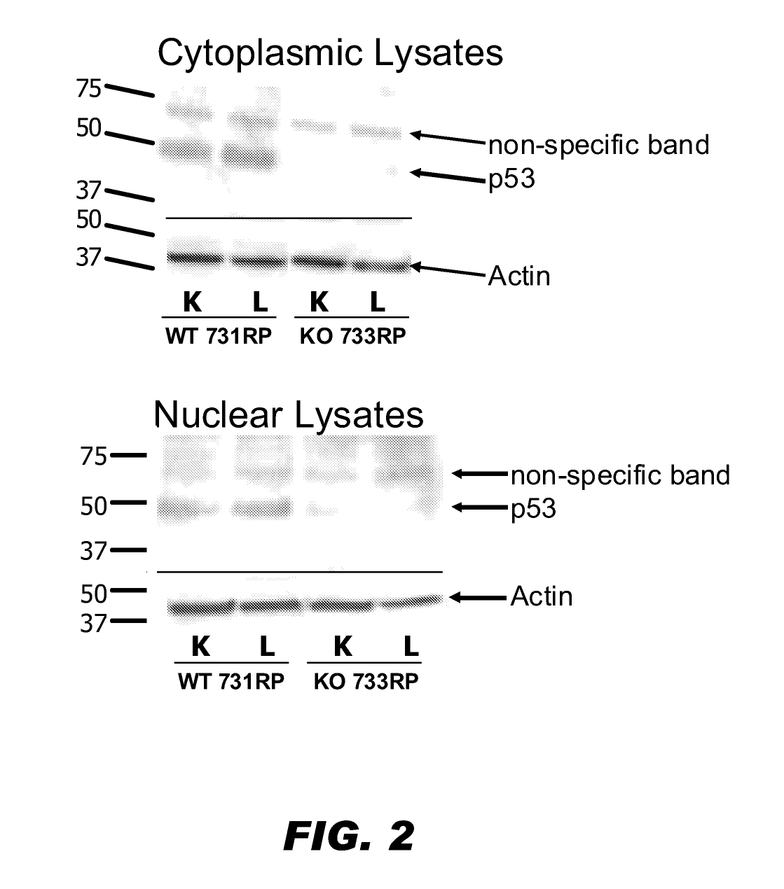

FIG. 2 illustrates knockout of the p53 gene in rats. Presented are Western blots of cytoplasmic and nuclear lysates of kidney (K) and liver (L) samples from wild-type (WT 731RP) and p53 knockout (KO 733RP) animals. The relative locations p53 protein and actin protein are indicated to the right of each image.

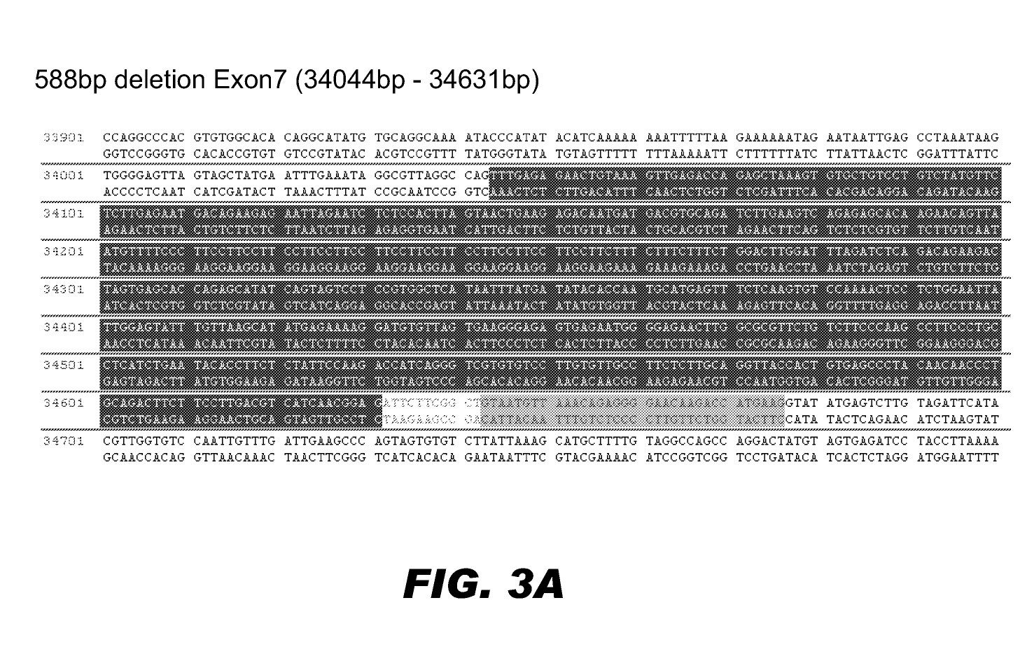

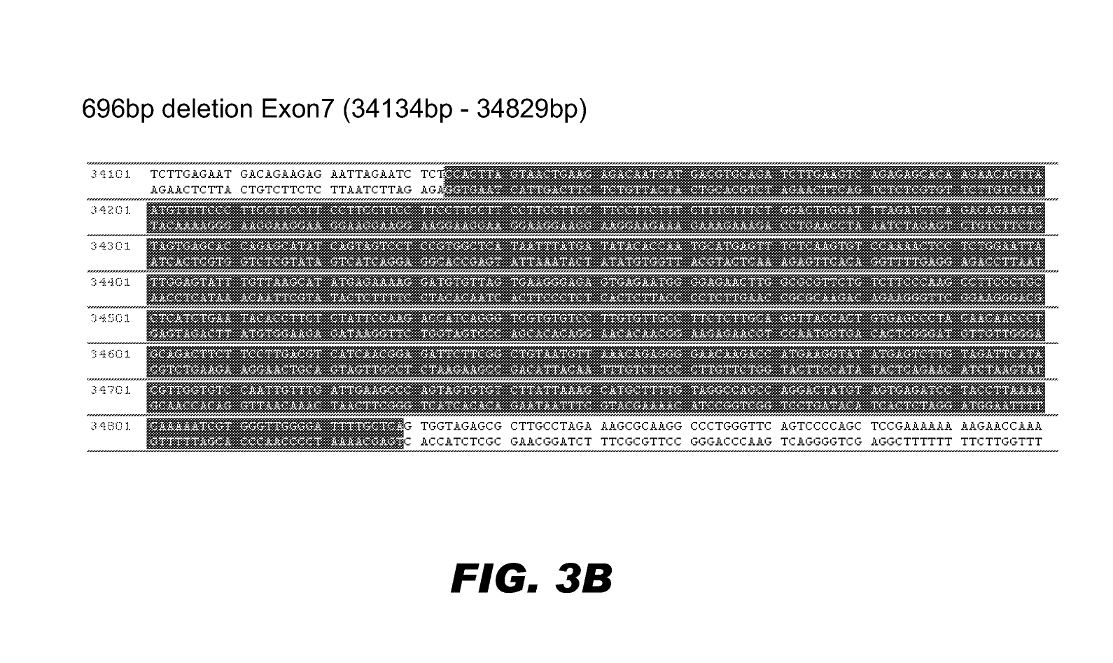

FIG. 3 presents the DNA sequences of edited BCRP loci in two animals. (A) Shows a region of the rat BCRP locus (SEQ ID NO:1) comprising a 588 bp deletion in exon 7. (B) Presents a region of the rat BCRP locus (SEQ ID NO:2) comprising a 696 bp deletion in exon 7. The exon sequence is shown in green; the target site is presented in yellow, and the deletions are shown in dark blue.

### DETAILED DESCRIPTION OF THE INVENTION

The present disclosure provides a genetically modified animal or animal cell comprising at least one edited chromosomal sequence encoding a protein involved in tumor suppression. The edited chromosomal sequence may be (1) inactivated, (2) modified, or (3) comprise an integrated sequence. An inactivated chromosomal sequence is altered such that a functional protein is not made. Thus, a genetically modified animal comprising an inactivated chromosomal sequence may be termed a “knock-out” or a “conditional knock-out.” Similarly, a genetically modified animal comprising an integrated sequence may be termed a “knock-in” or a “conditional knock-in.” As detailed below, a knock-in animal may be a humanized animal. Furthermore, a genetically modified animal comprising a modified chromosomal sequence may comprise a targeted point mutation(s) or other modification such that an altered protein product is produced. The chromosomal sequence encoding a protein involved in tumor suppression generally is edited using a zinc finger nuclease-mediated process. Briefly, the process comprises introducing into an embryo or cell at least one RNA molecule encoding a targeted zinc finger nuclease and, optionally, at least one accessory polynucleotide. The method further comprises incubating the embryo or cell to allow expression of the zinc finger nuclease, wherein a double-stranded break introduced into the targeted chromosomal sequence by the zinc finger nuclease is repaired by an error-prone non-homologous end-joining DNA repair process or a homology-directed DNA repair process. The method of editing chromosomal sequences involved in tumor suppression using targeted zinc finger nuclease technology is rapid, precise, and highly efficient.

### (I) Genetically Modified Animals

One aspect of the present disclosure provides a genetically modified animal in which at least one chromosomal sequence associated with tumor suppression has been edited. For example, the edited chromosomal sequence may be inactivated such that the sequence is not transcribed and/or a functional protein is not produced. Alternatively, the chromosomal sequence may be edited such that the regulation of expression of the protein is altered. For instance, the chromosomal sequence may be modified such that the protein associated with tumor suppression is over-produced. The edited chromosomal sequence may also be modified such that it codes for an altered tumor suppressor protein. For example, the chromosomal sequence may be modified such that at least one nucleotide is changed and the expressed protein comprises at least one changed amino acid residue (i.e., comprises a missense mutation). Furthermore, the edited chromosomal sequence may comprise a chromosomally integrated sequence encoding a protein associated with tumor suppression. The chromosomally integrated sequence may encode an endogenous protein associated with tumor suppression normally found in the animal, or the integrated sequence may encode an exogenous orthologous protein associated with tumor suppression, or combinations of both. The genetically modified animal disclosed herein may be heterozygous for the edited chromosomal sequence. Alternatively, the genetically modified animal may be homozygous for the edited chromosomal sequence.

In one embodiment, the genetically modified animal may comprise at least one inactivated chromosomal sequence encoding a protein associated with tumor suppression. The inactivated chromosomal sequence may include a deletion mutation (i.e., deletion of one or more nucleotides), an insertion mutation (i.e., insertion of one or more nucleotides), or a point mutation (i.e., substitution of a single nucleotide for another nucleotide). The deletion, insertion, or point mutation may lead to frame shift and/or splice site mutations such that at least one premature stop codon is introduced. As a consequence of the mutation, the targeted chromosomal sequence is inactivated and a functional protein is not produced. The inactivated chromosomal sequence comprises no exogenously introduced sequence. Such an animal may be termed a “knock-out.” Also included herein are genetically modified animals in which two, three, or more chromosomal sequences encoding proteins associated with tumor suppression are inactivated.

In another embodiment, the edited chromosomal sequence may be modified such that it codes for an altered protein associated with tumor suppression. The chromosomal sequence may be modified such that at least one nucleotide is changed and the expressed protein comprises at least one changed amino acid residue (i.e., a missense mutation). The chromosomal sequence may be modified to comprise more than one missense mutation such that more than one amino acid is changed. Additionally, the chromosomal sequence may be modified to have a three nucleotide deletion or insertion such that the expressed protein comprises a single amino acid deletion or insertion, provided such a protein is functional. The modified protein associated with tumor suppression may have altered substrate specificity, altered enzyme activity, altered kinetic rates, and so forth. In some embodiments, the modified protein comprises at least one modification such that the altered version of the protein provides tumor suppression activity. In other embodiments, the modified protein comprises at least one modification such that the altered version of the protein attenuates tumor suppression.

In a further embodiment, the genetically modified animal may comprise at least one chromosomally integrated sequence encoding a protein associated with tumor suppression. Integration of the sequence encoding a protein associated with tumor suppression may be random or it may be targeted. For example, an exogenous sequence encoding an orthologous or an endogenous protein associated with tumor suppression may be integrated into a chromosomal sequence encoding a protein associated with tumor suppression (or another protein) such that the endogenous chromosomal sequence is inactivated, but wherein the exogenous sequence encoding the orthologous or endogenous protein may be expressed. In such a case, the sequence encoding the orthologous or endogenous protein may be operably linked to an endogenous promoter control sequence. Alternatively, the promoter control sequence may be native to the exogenous sequence or the exogenous sequence may be operably linked to a heterologous promoter control sequence. The exogenous sequence encoding the orthologous or endogenous protein may be such that the protein associated with tumor suppression is over-produced, or the tissue-specific or temporal expression of the protein is altered, or a combination thereof. Alternatively, an exogenous sequence encoding an orthologous or endogenous protein may be integrated into a chromosomal sequence without affecting expression of an endogenous chromosomal sequence. For example, an exogenous sequence encoding a protein associated with tumor suppression may be integrated into a “safe harbor” locus, such as the Rosa26 locus, HPRT locus, or AAVS1 locus, wherein the exogenous sequence encoding the orthologous or endogenous protein may be expressed or over-expressed. An animal comprising a chromosomally integrated sequence encoding a protein associated with tumor suppression may be called a “knock-in,” and it should be understood that in such an iteration of the animal, no selectable marker is present. The sequence encoding a protein associated with tumor suppression also may be modified to include a tag or reporter. Suitable reporters include selectable markers such as cloramphenicol acetyltransferase (CAT) and neomycin phosphotransferase (neo), and fluorescent proteins such as green fluorescent protein (GFP), red fluorescent protein, or any genetically engineered variant thereof that improves the reporter performance. Non-limiting examples of known such FP variants include EGFP, blue fluorescent protein (EBFP, EBFP2, Azurite, mKalama1), cyan fluorescent protein (ECFP, Cerulean, CyPet) and yellow fluorescent protein derivatives (YFP, Citrine, Venus, YPet).

The chromosomally integrated sequence encoding a protein associated with tumor suppression may encode the wild-type form of the protein. Alternatively, the chromosomally integrated sequence encoding a protein associated with tumor suppression may comprise at least one modification such that an altered version of the protein is produced. In some embodiments, the chromosomally integrated sequence encoding a protein associated with tumor suppression comprises at least one modification such that the altered version of the protein enhances tumor suppression. In other embodiments, the chromosomally integrated sequence encoding a protein associated with tumor suppression comprises at least one modification such that the altered version of the protein attenuates tumor suppression.

In an additional embodiment, the genetically modified animal may be a “humanized” animal comprising at least one chromosomally integrated sequence encoding a functional tumor suppressor protein. The functional human protein may have no corresponding ortholog in the genetically modified animal. Alternatively, the wild-type animal from which the genetically modified animal is derived may comprise an ortholog corresponding to the human tumor suppressor protein. In this case, the orthologous sequence in the “humanized” animal is inactivated such that no functional protein is made and the “humanized” animal comprises at least one chromosomally integrated sequence encoding the human tumor suppressor protein. For example, a humanized animal may comprise an inactivated p53 sequence and a chromosomally integrated sequence encoding human p53 protein. Those of skill in the art appreciate that “humanized” animals may be generated by crossing a knock-out animal with a knock-in animal comprising the chromosomally integrated sequence.

In yet another embodiment, the genetically modified animal may comprise at least one edited chromosomal sequence encoding a protein associated with tumor suppression such that the expression pattern of the protein associated with tumor suppression is altered. For example, regulatory regions controlling the expression of the protein, such as a promoter or transcription binding site, may be altered such that the protein associated with tumor suppression is over-produced, or the tissue-specific or temporal expression of the protein is altered, or a combination thereof. Alternatively, the expression pattern of the protein associated with tumor suppression may be altered using a conditional knockout system. A non-limiting example of a conditional knockout system includes a Cre-lox recombination system. A Cre-lox recombination system comprises a Cre recombinase enzyme, a site-specific DNA recombinase that can catalyze the recombination of a nucleic acid sequence between specific sites (lox sites) in a nucleic acid molecule. Methods of using this system to produce temporal and tissue specific expression are known in the art. In general, a genetically modified animal is generated with lox sites flanking a chromosomal sequence, such as a chromosomal sequence encoding a protein associated with tumor suppression. The genetically modified animal comprising the lox-flanked chromosomal sequence encoding a protein associated with tumor suppression may then be crossed with another genetically modified animal expressing Cre recombinase. Progeny animals comprising the lox-flanked chromosomal sequence and the Cre recombinase are then produced, and the lox-flanked chromosomal sequence encoding a protein associated with tumor suppression is recombined, leading to deletion or inversion of the chromosomal sequence encoding a protein associated with tumor suppression. Expression of Cre recombinase may be temporally and conditionally regulated to effect temporally and conditionally regulated recombination of the chromosomal sequence encoding a protein associated with tumor suppression.

### (a) Chromosomal Sequences and Proteins Involved in Tumor Suppression

Tumor suppression genes are genes whose protein products protect a cell from one step on the path to cancer. A mutation in a tumor suppressor gene may cause a loss or reduction in the protective function of its protein product, thereby increasing the probability that a tumor will form, leading to cancer, usually in combination with other genetic changes. The proteins encoded by tumor suppressor genes have a dampening or repressive effect on the regulation of the cell cycle or promote apoptosis, and sometimes both. Tumor suppressor proteins are involved in the repression of genes essential for the continuing cell cycle; coupling the cell cycle to DNA damage so that the cell cycle can continue; initiating apoptosis in the cell if the damage cannot be repaired; and cell adhesion to prevent tumors from dispersing, blocking a loss of contact inhibition, and inhibiting metastasis.

Mutations in tumor suppressor genes can lead to various types of cancer, including but not limited to Retinoblastoma, Human Papilloma Virus, Wilms Tumor, Neurofibromatosis Type 1, Neurofibromatosis Type 2, familial adenomatous polyposis, Colon Cancer, Von Hippel-Lindau syndrome, Li-Fraumeni Syndrome, Familial Juvenile Polyposis syndrome, Familial Breast Cancer, Cowden Syndrome, Peutz-Jeghers Syndrome, Hereditary Nonpolyposis Colon Cancer Type 1, Hereditary Nonpolyposis Colon Cancer Type 2, Familial diffuse-type Gastic Cancer, Familial Melanoma, Gorlin Syndrome, Multiple Endocrine Neoplasia Type 1, and other tumor-related diseases.

The present disclosure comprises editing of any chromosomal sequences that encodes a protein associated with tumor suppression. The proteins associated with tumor suppression are typically selected based on an experimental association of the protein of interest with a cancer. For example, the production rate or circulating concentration of a protein associated with tumor suppression may be elevated or depressed in a population having cancer relative to a population not having cancer. Differences in protein levels may be assessed using proteomic techniques including but not limited to Western blot, immunohistochemical staining, enzyme linked immunosorbent assay (ELISA), and mass spectrometry. Alternatively, the proteins associated with tumor suppression may be identified by obtaining gene expression profiles of the genes encoding the proteins using genomic techniques including but not limited to DNA microarray analysis, serial analysis of gene expression (SAGE), and quantitative real-time polymerase chain reaction (Q-PCR).

By way of example, proteins involved in tumor suppression and their encoding chromosomal sequences may comprise, but is not limited to, TNF (tumor necrosis factor (TNF superfamily, member 2)), TP53 (tumor protein p53), ERBB2 (v-erb-b2 erythroblastic leukemia viral oncogene homolog 2, neuro/glioblastoma derived oncogene homolog (avian)), FN1 (fibronectin 1), TSC1 (tuberous sclerosis 1), PTGS2 (prostaglandin-endoperoxide synthase 2 (prostaglandin G/H synthase and cyclooxygenase)), PTEN (phosphatase and tensin homolog), PCNA (proliferating cell nuclear antigen), COL18A1 (collagen, type XVIII, alpha 1), TSSC4 (tumor suppressing subtransferable candidate 4), JUN (jun oncogene), MAPK8 (mitogen-activated protein kinase 8), TGFB1 (transforming growth factor, beta 1), IL6 (interleukin 6 (interferon, beta 2)), IFNG (interferon, gamma), BRCA1 (breast cancer 1, early onset), TSPAN32 (tetraspanin 32), BCL2 (B-cell CLL/lymphoma 2), NF2 (neurofibromin 2 (merlin)), GJB1 (gap junction protein, beta 1, 32 kDa), MAPK1 (mitogen-activated protein kinase 1), CD44 (CD44 molecule (Indian blood group)), PGR (progesterone receptor), TNS1 (tensin 1), PROK1 (prokineticin 1), SIAH1 (seven in absentia homolog 1 (Drosophila)), ENG (endoglin), TP73 (tumor protein p73), APC (adenomatous polyposis coli), BAX (BCL2-associated X protein), SRC (v-src sarcoma (Schmidt-Ruppin A-2) viral oncogene homolog (avian)), VHL (von Hippel-Lindau tumor suppressor), FHIT (fragile histidine triad gene), NFKB1 (nuclear factor of kappa light polypeptide gene enhancer in B-cells 1), IFNA1 (interferon, alpha 1), TGFBR1 (transforming growth factor, beta receptor 1), PRKCD (protein kinase C, delta), TGIF1 (TGFB-induced factor homeobox 1), DLC1 (deleted in liver cancer 1), SLC22A18 (solute carrier family 22, member 18), VEGFA (vascular endothelial growth factor A), MME (membrane metallo-endopeptidase), IL3 (interleukin 3 (colony-stimulating factor, multiple)), MKI67 (antigen identified by monoclonal antibody Ki-67), HSPD1 (heat shock 60 kDa protein 1 (chaperonin)), HSPB1 (heat shock 27 kDa protein 1), HSP90B2P (heat shock protein 90 kDa beta (Grp94), member 2 (pseudogene)), MBL2 (mannose-binding lectin (protein C) 2, soluble (opsonic defect)), ZFYVE9 (zinc finger, FYVE domain containing 9), TERT (telomerase reverse transcriptase), PML (promyelocytic leukemia), SKP2 (S-phase kinase-associated protein 2 (p45)), CYCS (cytochrome c, somatic), MAPK10 (mitogen-activated protein kinase 10), PAX7 (paired box 7), YAP1 (Yes-associated protein 1), PARP1 (poly (ADP-ribose) polymerase 1), MIR34A (microRNA 34a), PRKCA (protein kinase C, alpha), FAS (Fas (TNF receptor superfamily, member 6)), SYK (spleen tyrosine kinase), GSK3B (glycogen synthase kinase 3 beta), PRKCE (protein kinase C, epsilon), CYP19A1 (cytochrome P450, family 19, subfamily A, polypeptide 1), ABCB1 (ATP-binding cassette, sub-family B (MDR/TAP), member 1), NFKBIA (nuclear factor of kappa light polypeptide gene enhancer in B-cells inhibitor, alpha), RUNX1 (runt-related transcription factor 1), PRKCG (protein kinase C, gamma), RELA (v-rel reticuloendotheliosis viral oncogene homolog A (avian)), PLAU (plasminogen activator, urokinase), BTK (Bruton agammaglobulinemia tyrosine kinase), PRKCB (protein kinase C, beta), CSF1 (colony stimulating factor 1 (macrophage)), POMC (proopiomelanocortin), CEBPB (CCAAT/enhancer binding protein (C/EBP), beta), ROCK1 (Rho-associated, coiled-coil containing protein kinase 1), KDR (kinase insert domain receptor (a type III receptor tyrosine kinase)), NPM1 (nucleophosmin (nucleolar phosphoprotein B23, numatrin)), ROCK2 (Rho-associated, coiled-coil containing protein kinase 2), PRKAB1 (protein kinase, AMP-activated, beta 1 non-catalytic subunit), BAK1 (BCL2-antagonist/killer 1), AURKA (aurora kinase A), NTN1 (netrin 1), FLT1 (fms-related tyrosine kinase 1 (vascular endothelial growth factor/vascular permeability factor receptor)), NBN (nibrin), DNM3 (dynamin 3), PRDM10 (PR domain containing 10), PAX5 (paired box 5), EIF4G1 (eukaryotic translation initiation factor 4 gamma, 1), KAT2B (K(lysine)acetyltransferase 2B), TIMP3 (TIMP metallopeptidase inhibitor 3), CCL22 (chemokine (C—C motif) ligand 22), GRIN2B (glutamate receptor, ionotropic, N-methyl D-aspartate 2B), CD81 (CD81 molecule), CCL27 (chemokine (C—C motif) ligand 27), MAPK11 (mitogen-activated protein kinase 11), DKK1 (dickkopf homolog 1 (Xenopus laevis)), HYAL1 (hyaluronoglucosaminidase 1), CTSL1 (cathepsin L1), PKD1 (polycystic kidney disease 1 (autosomal dominant)), BUB1B (budding uninhibited by benzimidazoles 1 homolog beta (yeast)), MPP1 (membrane protein, palmitoylated 1, 55 kDa), SIAH2 (seven in absentia homolog 2 (Drosophila)), DUSP13 (dual specificity phosphatase 13), CCL21 (chemokine (C—C motif) ligand 21), RTN4 (reticulon 4), SMO (smoothened homolog (Drosophila)), CCL19 (chemokine (C—C motif) ligand 19), CSTF2 (cleavage stimulation factor, 3\′ pre-RNA, subunit 2, 64 kDa), RSF1 (remodeling and spacing factor 1), EZH2 (enhancer of zeste homolog 2 (Drosophila)), AK1 (adenylate kinase 1), CKM (creatine kinase, muscle), HYAL3 (hyaluronoglucosaminidase 3), ALOX15B (arachidonate 15-lipoxygenase, type B), PAG1 (phosphoprotein associated with glycosphingolipid microdomains 1), MIR21 (microRNA 21), S100A2 (S100 calcium binding protein A2), HYAL2 (hyaluronoglucosaminidase 2), CSTF1 (cleavage stimulation factor, 3\′ pre-RNA, subunit 1, 50 kDa), PCGF2 (polycomb group ring finger 2), THSD1 (thrombospondin, type I, domain containing 1), HOPX (HOP homeobox), SLC5A8 (solute carrier family 5 (iodide transporter), member 8), EMB (embigin homolog (mouse)), PAX9 (paired box 9), ARMCX3 (armadillo repeat containing, X-linked 3), ARMCX2 (armadillo repeat containing, X-linked 2), ARMCX1 (armadillo repeat containing, X-linked 1), RASSF4 (Ras association (RaIGDS/AF-6) domain family member 4), MIR34B (microRNA 34b), MIR205 (microRNA 205), RB1 (retinoblastoma 1), DYT10 (dystonia 10), CDKN2A (cyclin-dependent kinase inhibitor 2A (melanoma, p16, inhibits CDK4)), CDKN1A (cyclin-dependent kinase inhibitor 1A (p21, Cip1)), CCND1 (cyclin D1), AKT1 (v-akt murine thymoma viral oncogene homolog 1), MYC (v-myc myelocytomatosis viral oncogene homolog (avian)), CTNNB1 (catenin (cadherin-associated protein), beta 1, 88 kDa), MDM2 (Mdm2 p53 binding protein homolog (mouse)), SERPINB5 (serpin peptidase inhibitor, clade B (ovalbumin), member 5), EGF (epidermal growth factor (beta-urogastrone)), FOS (FBJ murine osteosarcoma viral oncogene homolog), NOS2 (nitric oxide synthase 2, inducible), CDK4 (cyclin-dependent kinase 4), SOD2 (superoxide dismutase 2, mitochondrial), SMAD3 (SMAD family member 3), CDKN1B (cyclin-dependent kinase inhibitor 1B (p27, Kip1)), SOD1 (superoxide dismutase 1, soluble), CCNA2 (cyclin A2), LOX (lysyl oxidase), SMAD4 (SMAD family member 4), HGF (hepatocyte growth factor (hepapoietin A; scatter factor)), THBS1 (thrombospondin 1), CDK6 (cyclin-dependent kinase 6), ATM (ataxia telangiectasia mutated), STAT3 (signal transducer and activator of transcription 3 (acute-phase response factor)), HIF1A (hypoxia inducible factor 1, alpha subunit (basic helix-loop-helix transcription factor)), IGF1R (insulin-like growth factor 1 receptor), MTOR (mechanistic target of rapamycin (serine/threonine kinase)), TSC2 (tuberous sclerosis 2), CDC42 (cell division cycle 42 (GTP binding protein, 25 kDa)), ODC1 (ornithine decarboxylase 1), SPARC (secreted protein, acidic, cysteine-rich (osteonectin)), HDAC1 (histone deacetylase 1), CDK2 (cyclin-dependent kinase 2), BARD1 (BRCA1 associated RING domain 1), CDH1 (cadherin 1, type 1, E-cadherin (epithelial)), EGR1 (early growth response 1), INSR (insulin receptor), IRF1 (interferon regulatory factor 1), PHB (prohibitin), PXN (paxillin), HSPA4 (heat shock 70 kDa protein 4), TYR (tyrosinase (oculocutaneous albinism IA)), CAV1 (caveolin 1, caveolae protein, 22 kDa), CDKN2B (cyclin-dependent kinase inhibitor 2B (p15, inhibits CDK4)), FOXO3 (forkhead box O3), HDAC9 (histone deacetylase 9), FBXW7 (F-box and WD repeat domain containing 7), FOXO1 (forkhead box O1), E2F1 (E2F transcription factor 1), STK11 (serine/threonine kinase 11), BMP2 (bone morphogenetic protein 2), HSP90AA1 (heat shock protein 90 kDa alpha (cytosolic), class A member 1), HNF4A (hepatocyte nuclear factor 4, alpha), CAMK2G (calcium/calmodulin-dependent protein kinase II gamma), TP53BP1 (tumor protein p53 binding protein 1), CRYAB (crystallin, alpha B), HMGCR (3-hydroxy-3-methylglutaryl-Coenzyme A reductase), PLAUR (plasminogen activator, urokinase receptor), MCL1 (myeloid cell leukemia sequence 1 (BCL2-related)), NOTCH1 (Notch homolog 1, translocation-associated (Drosophila)), RASSF1 (Ras association (RaIGDS/AF-6) domain family member 1), GSN (gelsolin), CADM1 (cell adhesion molecule 1), ATF2 (activating transcription factor 2), IFNB1 (interferon, beta 1, fibroblast), DAPK1 (death-associated protein kinase 1), CHFR (checkpoint with forkhead and ring finger domains), KITLG (KIT ligand), NDUFA13 (NADH dehydrogenase (ubiquinone) 1 alpha subcomplex, 13), DPP4 (dipeptidyl-peptidase 4), GLB1 (galactosidase, beta 1), IKZF1 (IKAROS family zinc finger 1 (Ikaros)), ST5 (suppression of tumorigenicity 5), TGFA (transforming growth factor, alpha), EIF4EBP1 (eukaryotic translation initiation factor 4E binding protein 1), TGFBR2 (transforming growth factor, beta receptor II (70/80 kDa)), EIF2AK2 (eukaryotic translation initiation factor 2-alpha kinase 2), GJA1 (gap junction protein, alpha 1, 43 kDa), MYD88 (myeloid differentiation primary response gene (88)), IF127 (interferon, alpha-inducible protein 27), RBMX (RNA binding motif protein, X-linked), EPHA1 (EPH receptor A1), TWSG1 (twisted gastrulation homolog 1 (Drosophila)), H2AFX (H2A histone family, member X), LGALS3 (lectin, galactoside-binding, soluble, 3), MUC3A (mucin 3A, cell surface associated), ILK (integrin-linked kinase), APAF1 (apoptotic peptidase activating factor 1), MAOA (monoamine oxidase A), ERBB3 (v-erb-b2 erythroblastic leukemia viral oncogene homolog 3 (avian)), EIF2S1 (eukaryotic translation initiation factor 2, subunit 1 alpha, 35 kDa), PER2 (period homolog 2 (Drosophila)), IGFBP7 (insulin-like growth factor binding protein 7), KDM5B (lysine (K)-specific demethylase 5B), SMARCA4 (SWI/SNF related, matrix associated, actin dependent regulator of chromatin, subfamily a, member 4), NME1 (non-metastatic cells 1, protein (NM23A) expressed in), F2RL1 (coagulation factor II (thrombin) receptor-like 1), ZFP36 (zinc finger protein 36, C3H type, homolog (mouse)), HSPA8 (heat shock 70 kDa protein 8), WNTSA (wingless-type MMTV integration site family, member 5A), ITGB4 (integrin, beta 4), RARB (retinoic acid receptor, beta), VEGFC (vascular endothelial growth factor C), CCL20 (chemokine (C—C motif) ligand 20), EPHB2 (EPH receptor B2), CSNK2A1 (casein kinase 2, alpha 1 polypeptide), PSMD9 (proteasome (prosome, macropain) 26S subunit, non-ATPase, 9), SERPINB2 (serpin peptidase inhibitor, clade B (ovalbumin), member 2), RHOB (ras homolog gene family, member B), DUSP6 (dual specificity phosphatase 6), CDKN1C (cyclin-dependent kinase inhibitor 1C (p57, Kip2)), SLIT2 (slit homolog 2 (Drosophila)), CEACAM1 (carcinoembryonic antigen-related cell adhesion molecule 1 (biliary glycoprotein)), UBC (ubiquitin C), STS (steroid sulfatase (microsomal), isozyme S), FST (follistatin), KRT1 (keratin 1), EIF6 (eukaryotic translation initiation factor 6), JUP (junction plakoglobin), HDAC4 (histone deacetylase 4), NEDD4 (neural precursor cell expressed, developmentally down-regulated 4), KRT14 (keratin 14), GLI2 (GLI family zinc finger 2), MYH11 (myosin, heavy chain 11, smooth muscle), MAPKAPK5 (mitogen-activated protein kinase-activated protein kinase 5), MAD1L1 (MAD1 mitotic arrest deficient-like 1 (yeast)), TNFAIP3 (tumor necrosis factor, alpha-induced protein 3), WEE1 (WEE1 homolog (S. pombe)), BTRC (beta-transducin repeat containing), NKX3-1 (NK3 homeobox 1), GPC3 (glypican 3), CREB3 (cAMP responsive element binding protein 3), PLCB3 (phospholipase C, beta 3 (phosphatidylinositol-specific)), DMPK (dystrophia myotonica-protein kinase), BLNK (B-cell linker), PPIA (peptidylprolyl isomerase A (cyclophilin A)), DAB2 (disabled homolog 2, mitogen-responsive phosphoprotein (Drosophila)), KLF4 (Kruppel-like factor 4 (gut)), RUNX3 (runt-related transcription factor 3), FLG (filaggrin), IVL (involucrin), COTS (chaperonin containing TCP1, subunit 5 (epsilon)), LRPAP1 (low density lipoprotein receptor-related protein associated protein 1), IGF2R (insulin-like growth factor 2 receptor), PERI (period homolog 1 (Drosophila)), BIK (BCL2-interacting killer (apoptosis-inducing)), PSMC4 (proteasome (prosome, macropain) 26S subunit, ATPase, 4), USF2 (upstream transcription factor 2, c-fos interacting), GAS1 (growth arrest-specific 1), LAMP2 (lysosomal-associated membrane protein 2), PSMD10 (proteasome (prosome, macropain) 26S subunit, non-ATPase, 10), IL24 (interleukin 24), GADD45G (growth arrest and DNA-damage-inducible, gamma), ARHGAP1 (Rho GTPase activating protein 1), CLDN1 (claudin 1), ANXA7 (annexin A7), CHN1 (chimerin (chimaerin) 1), TXNIP (thioredoxin interacting protein), PEG3 (paternally expressed 3), EIF3A (eukaryotic translation initiation factor 3, subunit A), CASC5 (cancer susceptibility candidate 5), TCF4 (transcription factor 4), CSNK2A2 (casein kinase 2, alpha prime polypeptide), CSNK2B (casein kinase 2, beta polypeptide), CRY1 (cryptochrome 1 (photolyase-like)), CRY2 (cryptochrome 2 (photolyase-like)), EIF4G2 (eukaryotic translation initiation factor 4 gamma, 2), LOXL2 (lysyl oxidase-like 2), PSMD13 (proteasome (prosome, macropain) 26S subunit, non-ATPase, 13), ANP32A (acidic (leucine-rich) nuclear phosphoprotein 32 family, member A), COL4A3 (collagen, type IV, alpha 3 (Goodpasture antigen)), SCGB1A1 (secretoglobin, family 1A, member 1 (uteroglobin)), BNIP3L (BCL2/adenovirus E1B 19 kDa interacting protein 3-like), MCC (mutated in colorectal cancers), EFNB3 (ephrin-B3), RBBP8 (retinoblastoma binding protein 8), PALB2 (partner and localizer of BRCA2), HBP1 (HMG-box transcription factor 1), MRPL28 (mitochondrial ribosomal protein L28), KDM5A (lysine (K)-specific demethylase 5A), QSOX1 (quiescin Q6 sulfhydryl oxidase 1), ZFR (zinc finger RNA binding protein), MN1 (meningioma (disrupted in balanced translocation) 1), SMYD4 (SET and MYND domain containing 4), USP7 (ubiquitin specific peptidase 7 (herpes virus-associated)), STK4 (serine/threonine kinase 4), THY1 (Thy-1 cell surface antigen), PTPRG (protein tyrosine phosphatase, receptor type, G), E2F6 (E2F transcription factor 6), STX11 (syntaxin 11), CDC42BPA (CDC42 binding protein kinase alpha (DMPK-like)), MYOCD (myocardin), DAP (death-associated protein), LOXL1 (lysyl oxidase-like 1), RNF139 (ring finger protein 139), HTATIP2 (HIV-1 Tat interactive protein 2, 30 kDa), AIM1 (absent in melanoma 1), BCCIP (BRCA2 and CDKN1A interacting protein), LOXL4 (lysyl oxidase-like 4), WWC1 (WW and C2 domain containing 1), LOXL3 (lysyl oxidase-like 3), CENPN (centromere protein N), TNS4 (tensin 4), SIK1 (salt-inducible kinase 1), PCGF6 (polycomb group ring finger 6), PHLDA3 (pleckstrin homology-like domain, family A, member 3), IL32 (interleukin 32), LATS1 (LATS, large tumor suppressor, homolog 1 (Drosophila)), COMMD7 (COMM domain containing 7), CDHR2 (cadherin-related family member 2), LELP1 (late cornified envelope-like proline-rich 1), NCRNA00188 (non-protein coding RNA 188), and ENSG00000131023.

Exemplary non-limiting examples of tumor suppression proteins include ATM (ataxia telangiectasia mutated), ATR (ataxia telangiectasia and Rad3 related), EGFR (epidermal growth factor receptor), ERBB2 (v-erb-b2 erythroblastic leukemia viral oncogene homolog 2), ERBB3 (v-erb-b2 erythroblastic leukemia viral oncogene homolog 3), ERBB4 (v-erb-b2 erythroblastic leukemia viral oncogene homolog 4), Notch 1, Notch 2, Notch 3, Notch 4, ATK1 (v-akt murine thymoma viral oncogene homolog 1), ATK2 (v-akt murine thymoma viral oncogene homolog 2), ATK3 (v-akt murine thymoma viral oncogene homolog 3), HIF1a (hypoxia-inducible factor 1a), HIF3a (hypoxia-inducible factor 1a), Met (met pronto-oncogene), HRG (histidine-rich glycoprotein), Bc12, PPAR(alpha) (peroxisome proliferator-activated receptor alpha), Ppar(gamma) (peroxisome proliferator-activated receptor gamma), WT1 (Wilmus Tumor 1), FGF1R (fibroblast growth factor 1 receptor) , FGF2R (fibroblast growth factor 1 receptor), FGF3R (fibroblast growth factor 3 receptor), FGF4R (fibroblast growth factor 4 receptor), FGF5R (fibroblast growth factor 5 receptor), CDKN2a (cyclin-dependent kinase inhibitor 2A), APC (adenomatous polyposis coli), Rb1 (retinoblastoma 1), MEN1 (multiple endocrine neoplasia1), VHL (von-Hippel-Lindau tumor suppressor), BRCA1 (breast cancer 1), BRCA2 (breast cancer 2), AR (androgen receptor), TSG101 (tumor susceptibility gene 101), Igf1(insulin-like growth factor 1), Igf2 (insulin-like growth factor 2), Igf 1R (insulin-like growth factor 1 receptor), Igf 2R (insulin-like growth factor 2 receptor), Bax (BCL-2 associated X protein), CASP 1 (Caspase 1), CASP 2 (Caspase 2), CASP 3 (Caspase 3), CASP 4(Caspase 4), CASP 6 (Caspase 6), CASP 7(Caspase 7), CASP 8 (Caspase 8), CASP 9 (Caspase 9), CASP 12 (Caspase 12), Kras (v-Ki-ras2 Kirsten rate sarcoma viral oncogene homolog), PTEN (phosphate and tensin homolog), BCRP (breast cancer receptor protein), p53, and combinations thereof.

(i) ATM

ATM, or ataxia telangiectasia mutated, provides instructions for making a protein, primarily located in the nucleus, which helps control the rate at which cells grow and divide. Additionally, ATM plays an important role in the normal development and activity of several body systems, including the nervous system and the immune system. ATM protein assists cells in recognizing damaged or broken DNA strands and coordinates DNA repair by activating enzymes that repair breaks in the DNA. The stability of the cell's genetic information is maintained by the efficient repair of damaged DNA. The ATM protein is of great interest in cancer research because of its central role in cell division and DNA repair.

Mutations in the ATM gene have been shown to be involved in several conditions. Ataxia-telangiectasia is caused by one of several hundred mutations in the ATM gene. Those afflicted with this disorder have mutations in both copies of the ATM gene in a cell. Breast cancer has also been found to be associated with the ATM gene. Patients with at least one family member with ataxia-telangiectasia are thought to have an increased risk of developing breast cancer. The mutation in the ATM gene prevents many of the body's cells from correctly repairing damaged DNA. People who have only one copy of the ATM gene in each cell, due to a deletion, are also at an increased risk of developing breast cancer. Cells that are missing one copy of the ATM gene produce half of the normal amount of ATM protein which leads to inefficient repair of DNA damage, leading to the accumulation of mutations in other genes. This leads to the possibility of the development of cancerous tumors. It has also been shown that people who carry one mutated copy of the ATM gene in each cell may have an increased risk of developing several other types of cancers, including, stomach, bladder, pancreas, lung, and ovarian cancer.

(ii) ATR

ATR, or ataxia telangiectasia and Rad3 related, is a protein kinase. Mutations of the ATR gene are associated with Sekel syndrome, a rare autosomal recessive disorder characterized by growth retardation, microencephaly with mental retardation, and a characteristic “bird headed” facial appearance. ATR is thought to be involved in DNA replication and DNA repair.

EGFR

EGFR or epidermal growth factor receptor is a transmembrane glycoprotein that is a member of the protein kinase superfamily. This protein is a receptor for members of the epidermal growth factor family. EGFR is a cell surface protein that binds to epidermal growth factor. When EGFR binds to a ligand, receptor dimerization and tyrosine autophosphorylation is induced, leading to cell proliferation. Mutations in EGFR are associated with lung cancer.

(iv) ERBB2

ERBB2, or v-erb-b2 erythroblastic leukemia viral oncogene homolog 2, neuro/glioblastoma derived oncogene homolog, is commonly referred to as Her-2/neu. ERBB2 growth factor receptor is located on the cell surface, where it associates with similar receptors to form a complex. Growth factors bind to these similar receptors and trigger the receptor complex to relay signals inside the cell activating certain genes that promote cell growth. It is thought that ERBB2 plays a role in cell adhesion, cell specialization, and cell movement. Breast cancer is associated with the ERBB2 gene and amplification of the ERBB2 gene is found in about 25% of breast cancers. The mutations in the ERBB2 gene are somatic mutations, thus, they are not inherited. One mechanism of somatic mutations is where DNA replicates in preparation for cell division resulting in multiple copies of the gene on a chromosome. Tumors can form as a result of multiple gene copies. Amplification of ERBB2 has also been linked to other types of cancer including ovarian, brain, stomach, and lung cancers.

(v) ERBB3

ERBB3 is also known as v-erb-b2 erythroblastic leukemia viral oncogene homolog 3 is a member of the epidermal growth factor receptor family of receptor tyrosine kinases. Amplification of this gene and overexpression of the ERBB3 protein have been linked to numerous cancers, including prostate, bladder, and breast tumors. ERBB3 binds to and is activated by neuregulins and NTAK. Mutations and defects in the ERBB3 gene are the cause of the lethal congenital contracture syndrome type 2, also referred to as Israeli Bedouin multiple contracture syndrome type A. Israeli Bedouin multiple contracture syndrome type A is characterized by multiple joint contractures, anterior horn atrophy in the spinal chord, and a distended bladder.

(vi) ERBB4

ERBB4 is also known as v-erb-b2 erythroblastic leukemia viral oncogene homolog 3 is related to ERBB2 and ERBB3 and serves a similar function.

(vii) Notch 1

Notch 1 is a member of the Type 1 transmembrane protein family and shares structural characteristics including an extracellular domain consisting of multiple epidermal growth factor-like repeats (EGF) and an intracellular domain consisting of multiple, different domain types. Notch proteins play a role in a variety of developmental processes by affecting the cell fate decisions. Notch genes have a signaling network that is an intracellular pathway which regulates interaction between physically adjacent cells. Defects in Notch 1 are a cause of bicuspid aortic valve, a common defect in the aortic valve where three heart leaflets are present instead of two. In rare cases, mutations in this gene can lead to restricted blood flow resulting in hypoplastic left heart syndrome. Aortic valve disease and T-cell acute lymphoblastic leukemia are associated with mutations in the Notch 1 gene.

(viii) Notch 2

Notch 2 is related to Notch 1, as both proteins are members of the Notch family of receptor proteins. Notch 2 has five ligands: Jagged 1, Jagged 2, Delta-like 1, Delta-like 3, and Delta-like 4. The Notch 2 protein and its ligands send signals that are important prior to birth, with research indicating that signals triggered by the interaction between Notch 2 and its ligands contribute to the development of cells destined to be a part of the heart, liver, kidney, teeth, and other structures in the growing embryo. Notch 2 is involved in tissue repair after birth.

Mutations in Notch 2 have been associated with Alagille syndrome. It is hypothesized that the mutations in Notch 2 probably result in a protein that is abnormally small or folded into an incorrect three-dimensional shape. Disrupted signaling in the Notc h2 gene is believed to cause development issues in the heart, liver, kidney and other parts of the body, resulting in the signs and symptoms of Alagille syndrome. Mutations in Notch 2 may be somatic mutations. These somatic mutations may lead to extra copies of the mutated gene, increasing gene activity. This increase in gene activity may lead to uncontrolled cell growth and cell division in the immune system cells, causing tumors.

(ix) Notch 3

Notch 3 receptor protein is located on the surface of muscle cells that surround blood vessels. Notch 3 receptor protein is specific to the arteries; however, the protein is not present in veins. Signals are sent by Notch 3 to the nucleus of the cell such that particular genes are activated within vascular smooth muscle cells. The Notch 3 receptors are though to be essential for the maintenance of healthy muscle cells in the brain's arteries.

Notch 3 is thought to be responsible for cerebral autosomal dominant arteriopathy with subcortical infarcts and leukoencephalopathy (CADASIL) due to one of 150 known mutations in the gene. Almost all of the 150 mutations change a single amino acid in the Notch 3 receptor. Disruption of the function of Notch 3 may lead to apoptosis of cells and damage to vascular smooth muscle cells. This damage to the vascular smooth muscle tissue is thought to cause recurrent strokes and other symptoms of CADASIL.

(x) Notch 4

Notch 4 is related to Notch 1, Notch 2, and Notch 3 and serves a similar function.

(xi) AKT1

AKT1, or v-akt murine thymoma viral oncogene homolog 1, is a serine-threonine protein kinase that is catalytically inactive in serum-starved primary and immortalized fibroblasts. AKT1 and related AKT2 are activated by platelet-derived growth factor. Growth factor-induced neuronal survival is mediated by AKTs. Survival factors can suppress apoptosis in a transcription-independent manner by activating the serine/threonine kinase AKT1, which then phosphorylates and inactivates the components of the apoptosis machinery.

Mutations in AKT1 are associated with colorectal cancer and an increased susceptibility to ovarian cancer and familial breast-ovarian cancer type 1. Additionally, mutations in AKT1 have been linked to the susceptibility of schizophrenia.

(xii) AKT2

AKT2, or v-akt murine thymona viral oncogene homolog 2, belongs to the subfamily of serine threonine kinases containing SH2-like domains. AKT2 was shown to be amplified and overexpressed in ovarian cancer cell lines and primary ovarian tumors. The overexpression of the AKT2 protein contributes to a malignant phenotype of human ductal pancreatic cancers. The AKT2 gene and protein is indicated in diabetes mellitus.

(xiii) AKT3

AKT3, or v-akt murine thymona viral oncogene homolog 3, encodes the AKT3 protein. The AKT3 gene is related to the AKT1 and AKT2 genes and mutations in the AKT3 gene caused overexpression of the AKT3 protein.

(xiv) HIF1A and HIF1B

HIF1A, hypoxia-inducible factor 1, alpha subunit, and HIF1B, hypoxia-inducible factor 1, beta subunit, encode the HIF1A and HIF1B proteins, respectively. HIF is stable and initiates gene transcription under hypoxia, whereas in normoxia, interaction with the von Hippel-Lindau tumor suppressor protein leads to rapid degradation of the HIF1A protein. Polymorphisms in the HIF1A are associated with renal cell carcinoma phenotype. The use of immunohistochemical assessment of HIF1A can be used as a predictor of poor outcome may improve clinical decision making regarding adjuvant treatment of patients with lymph node negative breast carcinoma.

(xv) Met

Met, or met pronto-oncogene (hepatocyte growth factor receptor), is a hepatocyte growth factor receptor that has tyrosine-kinase activity. The activation of MET after rearrangement with the TPR gene produces an oncogenic protein. MET has also been associated with gastric cancer, hepatocellular carcinoma, hereditary papillary renal carcinoma, and other related conditions. Genetic variations and mutations in MET may also be associated with susceptibility to autism type 9.

(xvi) HRG

HRG, or histidine-rich glycoprotein, contains two cysteine-like domains and is located in plasma and platelets. HRG can inhibit rosette formation and interacts with heparin, thrombospondin and plasminogen. There is a potential prothrombotic effect as exhibited by HRG's inhibition of fibrinolysis and the reduction of inhibition of coagulation. Mutations in HRG are thought to be the cause of thrombophilia due to histidine-rich glycoprotein deficiency.

(xvii) Bc12

Bc12 is a tumor suppressor protein related to p53. As a tumor suppressor, Bc12 is involved in the replication of cells. Mutations in Bc12 can lead to uncontrolled or abnormal cell replication.

(xviii) Ppar(alpha)

Ppar(alpha), or peroxisome proliferator-activated receptor alpha, is a nuclear receptor protein. Peroxisome proliferators include hypolipidemic drugs, herbicides, leukotriene antagonists, and plasticizers. Peroxisomes are subcellular organelles found in plants and animals that contain enzymes for respiration and for cholesterol and lipid metabolism. The action of peroxisomes is thought to be mediated via specific receptors, called PPARs, which affect the expression of target genes involved in cell proliferation, cell differentiation and in immune and inflammatory responses. Mutations in Ppar(alpha) have been linked to the susceptibility to hyperapobetalipoproteinemia.

(xix) Ppar(gamma)

Ppar(gamma), or peroxisome proliferator-activated receptor gamma, is another nuclear receptor. Ppar(gamma) regulates adipocyte differentiation that has been implicated in the pathology of numerous diseases such as obesity, diabetes, atherosclerosis, familial partial lipodystrophy type 3, and cancer. Mutations in Ppar(gamma) are associated with carotid intimal medial thickness 1. Other genetic variations in PPARG can be associated with susceptibility to glioma type 1. Gliomas are central nervous system neoplasms derived from glial cells and comprise astrocytomas, glioblastoma, multiforme, oligodendrogliomas, and ependymomas.

(xx) WT1

WT1, or Wilmus tumor 1, is a transcription factor that regulates the activity of genes involved in cell growth and apoptosis. Wilmus tumor is a rare form of kidney cancer, where the individual has a mutation in one copy of the WT1 gene in every cell. In other types of Wilmus tumor, WT1 gene mutations are only present in tumor cells. The mutations in the WT1 genes are somatic, meaning they are not inherited. Mutations in the WT1 gene have also been linked to several other forms of cancer including lung, prostate, breast, and ovarian cancer, as well as leukemia, such as acute lymphoblastic leukemia, chronic myeloid leukemia, and childhood acute myeloid leukemia. Denys-Drash syndrome is also caused by a mutation in the WT1 gene. WT1 mutations are also thought to cause Frasier syndrome. The mutations disrupt the way the WT1 gene's instructions are used to make the protein resulting in a shortage of functional protein.

(xxi) FGF Receptor Family

The FGF receptor family or fibroblast growth factor receptor family includes FGFR1, FGFR2, FGFR3, FGFR4, and FGFR5. FGF receptors are involved in important processes such as cell division, regulation of cell growth and maturation, formation of blood vessels, wound healing, and embryonic development. Growth factors work with the FGF receptor proteins to signal chemical reactions within the cell that instruct the cell to undergo certain changes, such as maturing to take on special functions.

The FGFR1 protein is thought to play a role in the development of the nervous system. Mutations in the FGFR1 gene are responsible for Kallmann syndrome, Pfeiffer syndrome, osteoglophonic dysplasia, and cancers such as pancreatic, esophageal, ovarian, testicular, breast, and head and neck cancers. Mutations in the FGFR2 gene have been lined to Apert syndrome, Beare-Stevenson cutis gyrate syndrome, Crouzon syndrome, Jackson-Weiss syndrome, Pfeiffer syndrome, lacrimo-auriculo-dento-digital (LADD) syndrome, and cancers such as prostate cancer, ovarian cancer, cervical cancer, pancreatic cancer, and head and neck cancers. FGFR3 gene mutations have been associated with Achondroplasia, Crouzonodermoskeletal syndrome, hypochondroplasia, Muenke syndrome, SADDAN, thanatophoric dysplasia, bladder cancer, and platyspondylic lethal skeletal dysplasia. Several types of cancers are associated with mutations in the FGFR4 gene, such as breast, colon, gastric, pancreatic, ovarian, head and neck, and prostate. These cancers are usually linked to a polymorphism in which glycine is replaced by arginine at position 338 in the protein's chain of amino acids.

(xxii) CDKN2a

CDKN2a, or cyclin-dependent kinase inhibitor 2A, encodes the CDKN2a protein. The CDKN2a gene generates several transcript variants which differ in their first exons, with at least three alternatively spliced variants encoding distinct proteins, two of which encode structurally related isoforms known to function as inhibitors of CDK4 kinase. This interaction with CDK4 and with CDK6 allows CDKN2a to act as a negative regulator of the proliferation of normal cells. Further, CDKN2a is capable of inducing cell cycle arrest in G1 and G2 phases and acts as a tumor suppressor. Genetic variations in CDKN2a may underlie susceptibility to uveal melanoma, which is the most common type of ocular malignant tumor. Mutations in CDKN2a are also linked to cutaneous malignant melanoma type 2, familiar atypical multiple mole melanoma-pancreatic carcinoma syndrome, melanoma-astrocytoma syndrome, Li Fraumeni syndrome, melanoma and neural system tumor syndrome, orolaryngeal cancer, pancreatic cancer, and melanoma syndrome.

(xxiii) APC

APC, is also referred to as adenomatous polyposis coli, plays a critical role in several cellular processes that determine whether a cell will develop into a tumor. The APC protein acts as a tumor suppressor, thus, it regulates the cell division cycle by keeping cells from growing and dividing too fast or in an uncontrolled way. Cell division, attachment and migration are all controlled, in part, by the APC protein. Mutations in the APC gene are associated with familial adenomatous polyposis, for which over 700 mutations have been identified. Cancers such as colorectal cancer, Turcot syndrome, colon cancer, and stomach cancer have been linked to mutations in the APC gene. Specifically, colon cancer has been linked to one mutation found in approximately 6% of people with Ashkenazi Jewish heritage that replaces isoleucine with lysine at position 1307 in the APC protein.

(xxiv) Rb1

Retinoblastoma 1, or Rb1, acts as a tumor suppressor, therefore, it regulates the cell cycle and prevents cells from dividing at a rapid pace or in an uncontrolled manner. The protein pRB can prevent other proteins from triggering DNA replication. Further, pRB interacts with other proteins to influence cell survival, apoptosis, and differentiation. Mutations in the RB1 gene have been associated with retinoblastoma, a rare type of eye cancer that typically affects young children. About 40% of retinoblastomas are germinal, and thus hereditary, while the other 60% of retinoblastomas are non-germinal, and thus, cannot be passed to the next generation. Bladder cancer, lung cancer, breast cancer, bone cancer, and melanoma have been associated with mutations in the RB1 gene.

(xxv) MEN1

MEN1 (menin), also referred to multiple endocrine neoplasia I, and acts as a tumor suppressor and is likely involved in several important cell functions. Copying and repairing DNA and apoptosis are functions in which menin plays a role. Menin is also present in the nucleus of many different cell types and appears to be active in all stages of development. Menin also interacts with several transcription factors which bind to specific areas of DNA to help control the expression of the genes. Mutations in MEN1 have been linked to multiple endocrine neoplasia, wherein over 400 mutations in MEN1 have been identified. Familial isolated hyperparathyroidism (FIHP) has also been associated with genetic variation in MEN, as well as sporadic tumors, tumors of the parathyroid gland, pancreatic tumors, and cancerous tumors of the airways in the lungs called bronchial carcinoids.

(xxvi) VHL

VHL, or von-Hippel-Lindau tumor suppressor, functions as a part of the VCB-CUL2 complex. This complex targets other proteins to be degraded by the cell when they are no longer required. This degradation removes damaged or unnecessary proteins and helps maintain the normal functions of cells. A protein called hypoxia-inducible factor (HIF) is targeted by the VCB-CUL2 complex to be broken down within cells. HIF controls several important genes involved in cell division and the formation of new blood vessels. VHL protein has also been thought to play a role in other cellular functions, including the regulation of other genes and control of cell division, as well as the formation of extracellular matrix. Mutations in the VHL gene have been linked to von Hippel-Lindau syndrome, wherein over 370 inherited mutations have been identified. The mutations cause an altered or missing VHL protein, leading to a build up of HIF in the cell, signaling the cell to divide abnormally and trigger the production of unnecessary blood vessels. Kidney cancer has also been linked to somatic mutations in VHL. VHL mutations are also linked to hemangioblastoma, Chuvash polycythemia or congenital polycythemia.

(xxvii) BRCA1

BRCA1, or breast cancer 1, early onset, is involved in repairing damaged DNA. BRCA1 protein interacts with several other proteins, including proteins produced by RAD51 and BARD1 genes, to mend breaks in DNA. By helping repair DNA, BRCA1 plays a role in maintaining the stability of a cell's genetic information. It is thought that BRCA1 plays a critical role in embryonic development also. Mutations in BRCA1 are most strongly linked to breast cancer, wherein over 1,000 mutations in the BRCA1 gene have been identified. Most of these over 1,000 mutations lead to the production of an abnormally short BRCA1 protein. Other mutations in BRCA1 change single amino acids in the protein or delete large segments of DNA from the BRCA1 gene. The mutations in BRCA1 often result in a protein that is unable to adequately repair damaged DNA or fix mutations that occur in other genes, allowing cells to divide in an uncontrolled manner, forming a tumor. Mutations in BRCA1 are also associated with an increased risk of fallopian tube cancer, male breast cancer, and pancreatic cancer.

(xxviii) BRCA2

BRCA2, or breast cancer 2, early onset, interacts with several other proteins, including RAD51 and BARD1, to mend breaks in DNA. By helping repair DNA, BRCA2 plays a role in maintaining the stability of a cell's genetic information. BRCA2 may also help regulate cytokinesis, a step in the cell division process where the cytoplasm divides to form two separate cells. BRCA2 is most strongly associated with breast cancer, wherein over 800 mutations in BRCA2 have been identified. These mutations insert or delete a small number of nucleotides in the gene, which disrupts protein production from one copy of the gene in each cell, resulting in an abnormally small, nonfunctional BRCA2 protein. BRCA2 mutations have also been associated with Fanconi anemia type D1, which results when two faulty copies of the BRCA2 gene are present in each cell. An increased risk of ovarian cancer, prostate cancer, pancreatic cancer, fallopian tube cancer, male breast cancer, and melanoma is associated with genetic variations in BRCA2.

(xxix) AR

AR, or androgen receptor, binds an androgen hormone to form an androgen-receptor complex that binds to DNA and regulates the activity of the androgen-responsive genes. Androgen receptors help direct the development of male sexual characteristics, as well as regulating hair growth and sex drive in females. Mutations in the AR gene have been known to cause androgen insensitivity syndrome. Most of the mutations leading to androgen insensitivity syndrome cause changes in single base pairs of DNA, sometimes leading to a shortened version of the AR protein or leading to an abnormal receptor that cannot bind to androgens or to DNA. Other disorders associated with genetic variation in the AR protein include spinal and bulbar muscular atrophy, androgenetic alopecia, breast cancer, prostate cancer, and endometrial cancer in women.

(xxx) TSG101

TSG101, or tumor susceptibility gene 101, belongs to a group of inactive homologs of ubiquitin-conjugating enzymes. The protein may play a role in cell growth and differentiation and acts as a negative growth regulator. Genomic stability and cell cycle regulation appear to be linked to the in vivo steady-state expression of TSG101, which acts as a tumor suppressor. Mutations in the TSG101 gene occur in high frequency in breast cancer. (xxxi) Igf1 and Igf2

Igf1 and Igf2, or insulin-like growth factor 1 and insulin-like growth factor 2, are structurally and functionally related to insulin but have a much higher growth-promoting activity. Igf2 is thought to play an essential role in growth and development before birth. With regard to the inheritance of Igf2, the copy of the gene inherited from a person's father is the only active copy in most parts of the body. Mutations in Igf1 have been linked to insulin-like growth factor 1 deficiency, an autosomal recessive disorder characterized by growth retardation, sensorineural deafness, and mental retardation. Mutations in Igf2 are associated with Beckwith-Wiedmann syndrome and an increased susceptibility to cancers such as Wilmus tumor, heptaoblastoma, embryonal tumors, Russell-Silver syndrome, breast cancer, prostate cancer, lung cancer, colon cancer, and liver cancer. Normal variations in the Igf2 gene may also be involved in determining the adult height and/or weight of an individual.

(xxxii) Iqf 1R and Iqf 2R

Igf 1R (insulin-like growth factor 1 receptor) and Igf 2R (insulin-like growth factor 2 receptor), bind insulin-like growth factors with a high affinity and possess tyrosine kinase activity. Igf 1R plays a critical role in transformation events. Igf 2R is a receptor for both insulin-like growth factor 2 and mannose 6-phosphate. Genetic variance in Igf 1R has been associated with a growth deficiency disorder characterized by intrauterine growth retardation and poor postnatal growth accompanied by increased plasma Igf1. Mutations in Igf 2R are associated with heptocellular carcinoma.

(xxxiii) Bax

Bax, or BCL-2 associated X protein, encodes for the BCL2 protein. BCL2 family member proteins form hetero-or homodimers and act as anti- or pro-apoptotic regulators that are involved in a variety of cellular activities. The expression of the Bax gene is regulated by the tumor suppressor p53 and has been shown to be involved in p53-mediated apoptosis. Mutations in the Bax gene have been associated with colorectal cancer and t-cell acute lymphoblastic leukemia.

(xxxiv) Caspase Family (1, 2, 3, 4, 6, 7, 8, 9, 12)

The caspase family: CASP1, CASP2, CASP3, CASP4, CASP6, CASP7, CASP8, CASP9, and CASP12; are a family of proteases responsible for carrying out the cell death process. The proteases are kept active by proteins on the mitochondrial cell surface from the BCL2 family. When a cell is exposed to cell death signals, such as ischemia, chemotherapy, or radiation, BCL2 function is blocked and caspase activators initiate the cell death cascade. CASP8, caspase 8, is associated with autoimmune lymphoproliferative syndrome, hepatocellular carcinoma, somatic, and lung cancer. CASP12 is associated with the susceptibility to sepsis.

(xxxv) Kras

Kras, or v-Ki-ras2 Kirsten rate sarcoma viral oncogene homolog, is primarily involved in regulating cell division. K-Ras uses signal transduction to relay signals from the outside of the cell into the cell's nucleus. The signals provided by K-Ras instruct the cell to grow, divide, and differentiate. K-Ras is a GTPase, meaning that it converts GTP to GDP, thus, K-Ras acts like a switch that is turned on and off by the GTP and GDP molecules. The Kras gene belongs to a class of genes known as oncogenes that, when mutated, have the potential to cause normal cells to become cancerous. Mutations in Kras have been linked to Noonan Syndrome, several types of cancers, including pancreatic, lung, and colorectal cancers, cardiofaciocutaneous syndrome, and Costello syndrome.

(xxxvi) Pten

Pten, or phosphate and tensin homolog, acts as a tumor suppressor, thus, helps regulate the cycle of cell division by keeping cells from growing and dividing too rapidly or in an uncontrolled way. PTEN modifies other proteins and lipids by removing phosphate groups. The PTEN enzyme acts as part of a chemical pathway that signals the cell to stop dividing and triggers cells to undergo a form of programmed cell death, called apoptosis. PTEN acts to control cell growth so that it does not become irregular or uncontrolled, leading to cancer. Mutations in the PTEN gene have been associated with Cowden syndrome, breast cancer, Bannayan-Riley-Rubalcaba syndrome, Proteus syndrome, and Proteus-like syndrome. These disorders are collectively referred to as PTEN hamartoma tumor syndromes (PHTS).

(xxxvii) BCRP

BCRP, or breast cancer resistance protein, has been linked to breast cancer, but its biological significance is largely unknown. BRCP is also known as ABCG2. BRCP is an ATP-binding transport protein that is expressed in several organs, including the liver. BCRP is an ABC transport protein and these transport proteins have been shown to play an important pathophysiological role in several liver diseases.

(xxxviii) p53

P53 is a nuclear localized phosphoprotein. P53 is thought to be involved in transcription regulation. Phosphorylation regulates the activity of p53 and the level of p53 is low after mitosis, but increases after G₁. Further, p53 may also regulate the initiation of DNA synthesis. Due to the involvement of p53 in both transcription and DNA replication, the various mutants of p53 may also regulate the initiation of DNA synthesis.

### (b) Animals

The term “animal,” as used herein, refers to a non-human animal. The animal may be an embryo, a juvenile, or an adult. Suitable animals include vertebrates such as mammals, birds, reptiles, amphibians, and fish. Examples of suitable mammals include without limit rodents, companion animals, livestock, and primates. Non-limiting examples of rodents include mice, rats, hamsters, gerbils, and guinea pigs. Suitable companion animals include but are not limited to cats, dogs, rabbits, hedgehogs, and ferrets. Non-limiting examples of livestock include horses, goats, sheep, swine, cattle, llamas, and alpacas. Suitable primates include, but are not limited to, capuchin monkeys, chimpanzees, lemurs, macaques, marmosets, tamarins, spider monkeys, squirrel monkeys, and vervet monkeys. Non-limiting examples of birds include chickens, turkeys, ducks, and geese. Alternatively, the animal may be an invertebrate such as an insect, a nematode, and the like. Non-limiting examples of insects include Drosophila and mosquitoes. An exemplary animal is a rat. Non-limiting examples of commonly used rat strains suitable for genetic manipulation include Dahl Salt-Sensitive, Fischer 344, Lewis, Long Evans Hooded, Sprague-Dawley and Wistar. In another iteration of the invention, the animal does not comprise a genetically modified mouse. In each of the foregoing iterations of suitable animals for the invention, the animal does not include exogenously introduced, randomly integrated transposon sequences.

The protein involved in tumor suppression may be endogenous to the animal or it may be exogenous (such as an orthologous tumor suppressor protein). Exogenous proteins may be from any of the animals listed above, as well as from human. The type of genetically modified animal and the source of the protein involved in tumor suppression can and will vary. As an example, the genetically modified animal may be a rat, cat, dog, or pig, and the protein involved in tumor suppression may be human. One of skill in the art will readily appreciate that numerous combinations are possible. In preferred embodiment, the animal is a rat and preferred sequences involved in tumor suppression are listed below. In embodiments in which the genetically modified animal comprises at least one chromosomally integrated sequence encoding an exogenous protein involved in tumor suppression, the exogenous protein is human. In an exemplary embodiment, the genetically modified animal is a rat and the exogenous protein involved in tumor suppression is human.

| ID | Ref Seq. |
|---|---|
| ATM | NM_001106821 |
| ATR | XR_086326 |
| EGFR | NM_031507 |
| ERBB2 | NM_017003 |
| ERBB3 | NM_017218 |
| ERBB4 | NM_021687 |
| Notch1 | NM_001105721 |
| Notch2 | NM_024358 |
| Notch3 | NM_020087 |
| Notch4 | NM_001002827 |
| AKT1 | NM_033230 |
| AKT2 | NM_017093 |
| AKT3 | NM_031575 |
| HIF1a, HIF3a | NM_024359, NM_022528 |
| Met | NM_031517 |
| HRG | NM_133428 |
| Bcl2 | NM_016993.1 |
| PPAR alpha | NM_013196 |
| PPAR gamma | NM_013124, NM_001145367, |
| | NM_001145366 |
| WT1 (Wilms Tumor) | NM_031534 |
| FGF Receptor Family | NM_199114, NM_012712, |
| (5 members: 1, 2, 3, 4, 5) | NM_001109892, NM_001109893, |
| | NM_001109894, NM_001109895, |
| | NM_001109896, NM_053429, |
| | NM_001109904, NM_199114 (transcript |
| | variants) |
| CDKN2a | NM_031550 |
| APC | NM_012499.1 |
| Rb1 (retinoblastoma) | NM_017045 |
| MEN1 | NM_019208 |
| VHL | NM_052801 |
| BRCA1 | NM_012514 |
| BRCA2 | NM_031542 |
| AR (Androgen Receptor) | NM_012502 |
| TSG101 | NM_181628 |
| Igf1 (4 variants) | NM_001082477, NM_178866, |
| | NM_001082478, NM_001082479 |
| Igf2 (3 variants) | NM_031511, NM_001190162, |
| | NM_001190163 |
| Igf 1 Receptor | NM_052807 |
| Igf 2 Receptor | NM_012756 |
| Bax | NM_017059 |
| Caspase Family | NM_012762, NM_022522, NM_012922, |
| (9 members: 1, 2, 3, 4, | NM_053736, NM_031775, |
| 6, 7, 8, 9, 12) | NM_022260, NM_022277, NM_031632, |
| | NM_130422 |
| Kras | NM_031515 |
| PTEN | NM_031606 |

### (II) Genetically Modified Cells

A further aspect of the present disclosure provides genetically modified cells or cell lines comprising at least one edited chromosomal sequence encoding a protein involved in tumor suppression. The genetically modified cell or cell line may be derived from any of the genetically modified animals disclosed herein. Alternatively, the chromosomal sequence involved in tumor suppression may be edited in a cell as detailed below. The disclosure also encompasses a lysate of said cells or cell lines.

In general, the cells will be eukaryotic cells. Suitable host cells include fungi or yeast, such as Pichia, Saccharomyces, or Schizosaccharomyces; insect cells, such as SF9 cells from Spodoptera frugiperda or S2 cells from Drosophila melanogaster; and animal cells, such as mouse, rat, hamster, non-human primate, or human cells. Exemplary cells are mammalian. The mammalian cells may be primary cells. In general, any primary cell that is sensitive to double strand breaks may be used. The cells may be of a variety of cell types, e.g., fibroblast, myoblast, T or B cell, macrophage, epithelial cell, and so forth.

When mammalian cell lines are used, the cell line may be any established cell line or a primary cell line that is not yet described. The cell line may be adherent or non-adherent, or the cell line may be grown under conditions that encourage adherent, non-adherent or organotypic growth using standard techniques known to individuals skilled in the art. Non-limiting examples of suitable mammalian cell lines include Chinese hamster ovary (CHO) cells, monkey kidney CVI line transformed by SV40 (COS7), human embryonic kidney line 293, baby hamster kidney cells (BHK), mouse sertoli cells (TM4), monkey kidney cells (CVI-76), African green monkey kidney cells (VERO), human cervical carcinoma cells (HeLa), canine kidney cells (MDCK), buffalo rat liver cells (BRL 3A), human lung cells (W138), human liver cells (Hep G2), mouse mammary tumor cells (MMT), rat hepatoma cells (HTC), HIH/3T3 cells, the human U2-OS osteosarcoma cell line, the human A549 cell line, the human K562 cell line, the human HEK293 cell lines, the human HEK293T cell line, and TRI cells. For an extensive list of mammalian cell lines, those of ordinary skill in the art may refer to the American Type Culture Collection catalog (ATCC®, Mamassas, Va.).

In still other embodiments, the cell may be a stem cell. Suitable stem cells include without limit embryonic stem cells, ES-like stem cells, fetal stem cells, adult stem cells, pluripotent stem cells, induced pluripotent stem cells, multipotent stem cells, oligopotent stem cells, and unipotent stem cells.

### (III) Zinc Finger-Mediated Genome Editing

In general, the genetically modified animal or cell detailed above in sections (I) and (II), respectively, is generated using a zinc finger nuclease-mediated genome editing process. The process for editing a chromosomal sequence comprises: (a) introducing into an embryo or cell at least one nucleic acid encoding a zinc finger nuclease that recognizes a target sequence in the chromosomal sequence and is able to cleave a site in the chromosomal sequence, and, optionally, (i) at least one donor polynucleotide comprising a sequence for integration flanked by an upstream sequence and a downstream sequence that share substantial sequence identity with either side of the cleavage site, or (ii) at least one exchange polynucleotide comprising a sequence that is substantially identical to a portion of the chromosomal sequence at the cleavage site and which further comprises at least one nucleotide change; and (b) culturing the embryo or cell to allow expression of the zinc finger nuclease such that the zinc finger nuclease introduces a double-stranded break into the chromosomal sequence, and wherein the double-stranded break is repaired by (i) a non-homologous end-joining repair process such that an inactivating mutation is introduced into the chromosomal sequence, or (ii) a homology-directed repair process such that the sequence in the donor polynucleotide is integrated into the chromosomal sequence or the sequence in the exchange polynucleotide is exchanged with the portion of the chromosomal sequence.

Components of the zinc finger nuclease-mediated method are described in more detail below.

### (a) Zinc Finger Nuclease

The method comprises, in part, introducing into an embryo or cell at least one nucleic acid encoding a zinc finger nuclease. Typically, a zinc finger nuclease comprises a DNA binding domain (i.e., zinc finger) and a cleavage domain (i.e., nuclease). The DNA binding and cleavage domains are described below. The nucleic acid encoding a zinc finger nuclease may comprise DNA or RNA. For example, the nucleic acid encoding a zinc finger nuclease may comprise mRNA. When the nucleic acid encoding a zinc finger nuclease comprises mRNA, the mRNA molecule may be 5′ capped. Similarly, when the nucleic acid encoding a zinc finger nuclease comprises mRNA, the mRNA molecule may be polyadenylated. An exemplary nucleic acid according to the method is a capped and polyadenylated mRNA molecule encoding a zinc finger nuclease. Methods for capping and polyadenylating mRNA are known in the art.

(i) Zinc Finger Binding Domain

Zinc finger binding domains may be engineered to recognize and bind to any nucleic acid sequence of choice. See, for example, Beerli et al. (2002) Nature Biotechnol. 20:135-141; Pabo et al. (2001) Ann. Rev. Biochem. 70:313-340; Isalan et al. (2001) Nature Biotechnol. 19:656-660; Segal et al. (2001) Curr. Opin. Biotechnol. 12:632-637; and Choo et al. (2000) Curr. Opin. Struct. Biol. 10:411-416. An engineered zinc finger binding domain may have a novel binding specificity compared to a naturally-occurring zinc finger protein. Engineering methods include, but are not limited to, rational design and various types of selection. Rational design includes, for example, using databases comprising doublet, triplet, and/or quadruplet nucleotide sequences and individual zinc finger amino acid sequences, in which each doublet, triplet or quadruplet nucleotide sequence is associated with one or more amino acid sequences of zinc fingers which bind the particular triplet or quadruplet sequence. See, for example, U.S. Pat. Nos. 6,453,242 and 6,534,261, the disclosures of which are incorporated by reference herein in their entireties. As an example, the algorithm of described in U.S. Pat. No. 6,453,242 may be used to design a zinc finger binding domain to target a preselected sequence. Alternative methods, such as rational design using a nondegenerate recognition code table may also be used to design a zinc finger binding domain to target a specific sequence (see, for example, Biochemistry 2002, 41, 7074-7081).

A zinc finger binding domain may be designed to recognize a DNA sequence ranging from about 3 nucleotides to about 21 nucleotides in length, or from about 8 to about 19 nucleotides in length. In general, the zinc finger binding domains of the zinc finger nucleases disclosed herein comprise at least three zinc finger recognition regions (i.e., zinc fingers). In one embodiment, the zinc finger binding domain may comprise four zinc finger recognition regions. In another embodiment, the zinc finger binding domain may comprise five zinc finger recognition regions. In still another embodiment, the zinc finger binding domain may comprise six zinc finger recognition regions. A zinc finger binding domain may be designed to bind to any suitable target DNA sequence. See for example, U.S. Pat. Nos. 6,607,882; 6,534,261 and 6,453,242, the disclosures of which are incorporated by reference herein in their entireties.

Exemplary methods of selecting a zinc finger recognition region may include phage display and two-hybrid systems, and are disclosed in U.S. Pat. Nos. 5,789,538; 5,925,523; 6,007,988; 6,013,453; 6,410,248; 6,140,466; 6,200,759; and 6,242,568; as well as WO 98/37186; WO 98/53057; WO 00/27878; WO 01/88197 and GB 2,338,237, each of which is incorporated by reference herein in its entirety. In addition, enhancement of binding specificity for zinc finger binding domains has been described, for example, in WO 02/077227.

Zinc finger binding domains and methods for design and construction of fusion proteins (and polynucleotides encoding same) are known to those of skill in the art and are described in detail in U.S. Patent Application Publication Nos. 20050064474 and 20060188987, each incorporated by reference herein in its entirety. Zinc finger recognition regions and/or multi-fingered zinc finger proteins may be linked together using suitable linker sequences, including for example, linkers of five or more amino acids in length. See, U.S. Pat. Nos. 6,479,626; 6,903,185; and 7,153,949, the disclosures of which are incorporated by reference herein in their entireties, for non-limiting examples of linker sequences of six or more amino acids in length. The zinc finger binding domain described herein may include a combination of suitable linkers between the individual zinc fingers of the protein.

In some embodiments, the zinc finger nuclease may further comprise a nuclear localization signal or sequence (NLS). A NLS is an amino acid sequence which facilitates targeting the zinc finger nuclease protein into the nucleus to introduce a double stranded break at the target sequence in the chromosome. Nuclear localization signals are known in the art. See, for example, Makkerh et al. (1996) Current Biology 6:1025-1027.

An exemplary zinc finger DNA binding domain recognizes and binds a sequence having at least about 80% sequence identity with a sequence chosen from SEQ ID NOs:3, 4, 5, 6, 7, and 8. In other embodiments, the sequence identity may be about 81%, 82%, 83%, 84%, 85%, 86%, 87%, 88%, 89%, 90%, 91%, 92%, 93%, 94%, 95%, 96%, 97%, 98%, 99%, or 100%.

(ii) Cleavage Domain

A zinc finger nuclease also includes a cleavage domain. The cleavage domain portion of the zinc finger nucleases disclosed herein may be obtained from any endonuclease or exonuclease. Non-limiting examples of endonucleases from which a cleavage domain may be derived include, but are not limited to, restriction endonucleases and homing endonucleases. See, for example, 2002-2003 Catalog, New England Biolabs, Beverly, Mass.; and Belfort et al. (1997) Nucleic Acids Res. 25:3379-3388 or www.neb.com. Additional enzymes that cleave DNA are known (e.g., S1 Nuclease; mung bean nuclease; pancreatic DNase I; micrococcal nuclease; yeast HO endonuclease). See also Linn et al. (eds.) Nucleases, Cold Spring Harbor Laboratory Press, 1993. One or more of these enzymes (or functional fragments thereof) may be used as a source of cleavage domains.

A cleavage domain also may be derived from an enzyme or portion thereof, as described above, that requires dimerization for cleavage activity. Two zinc finger nucleases may be required for cleavage, as each nuclease comprises a monomer of the active enzyme dimer. Alternatively, a single zinc finger nuclease may comprise both monomers to create an active enzyme dimer. As used herein, an “active enzyme dimer” is an enzyme dimer capable of cleaving a nucleic acid molecule. The two cleavage monomers may be derived from the same endonuclease (or functional fragments thereof), or each monomer may be derived from a different endonuclease (or functional fragments thereof).

When two cleavage monomers are used to form an active enzyme dimer, the recognition sites for the two zinc finger nucleases are preferably disposed such that binding of the two zinc finger nucleases to their respective recognition sites places the cleavage monomers in a spatial orientation to each other that allows the cleavage monomers to form an active enzyme dimer, e.g., by dimerizing. As a result, the near edges of the recognition sites may be separated by about 5 to about 18 nucleotides. For instance, the near edges may be separated by about 5, 6, 7, 8, 9, 10, 11, 12, 13, 14, 15, 16, 17 or 18 nucleotides. It will however be understood that any integral number of nucleotides or nucleotide pairs may intervene between two recognition sites (e.g., from about 2 to about 50 nucleotide pairs or more). The near edges of the recognition sites of the zinc finger nucleases, such as for example those described in detail herein, may be separated by 6 nucleotides. In general, the site of cleavage lies between the recognition sites.

Restriction endonucleases (restriction enzymes) are present in many species and are capable of sequence-specific binding to DNA (at a recognition site), and cleaving DNA at or near the site of binding. Certain restriction enzymes (e.g., Type IIS) cleave DNA at sites removed from the recognition site and have separable binding and cleavage domains. For example, the Type IIS enzyme Fok I catalyzes double-stranded cleavage of DNA, at 9 nucleotides from its recognition site on one strand and 13 nucleotides from its recognition site on the other. See, for example, U.S. Pat. Nos. 5,356,802; 5,436,150 and 5,487,994; as well as Li et al. (1992) Proc. Natl. Acad. Sci. USA 89:4275-4279; Li et al. (1993) Proc. Natl. Acad. Sci. USA 90:2764-2768; Kim et al. (1994a) Proc. Natl. Acad. Sci. USA 91:883-887; Kim et al. (1994b) J. Biol. Chem. 269:31, 978-31, 982. Thus, a zinc finger nuclease may comprise the cleavage domain from at least one Type IIS restriction enzyme and one or more zinc finger binding domains, which may or may not be engineered. Exemplary Type IIS restriction enzymes are described for example in International Publication WO 07/014,275, the disclosure of which is incorporated by reference herein in its entirety. Additional restriction enzymes also contain separable binding and cleavage domains, and these also are contemplated by the present disclosure. See, for example, Roberts et al. (2003) Nucleic Acids Res. 31:418-420.

An exemplary Type IIS restriction enzyme, whose cleavage domain is separable from the binding domain, is Fok I. This particular enzyme is active as a dimmer (Bitinaite et al. (1998) Proc. Natl. Acad. Sci. USA 95: 10, 570-10, 575). Accordingly, for the purposes of the present disclosure, the portion of the Fok I enzyme used in a zinc finger nuclease is considered a cleavage monomer. Thus, for targeted double-stranded cleavage using a Fok I cleavage domain, two zinc finger nucleases, each comprising a Fok I cleavage monomer, may be used to reconstitute an active enzyme dimer. Alternatively, a single polypeptide molecule containing a zinc finger binding domain and two Fok I cleavage monomers may also be used.

In certain embodiments, the cleavage domain may comprise one or more engineered cleavage monomers that minimize or prevent homodimerization, as described, for example, in U.S. Patent Publication Nos. 20050064474, 20060188987, and 20080131962, each of which is incorporated by reference herein in its entirety. By way of non-limiting example, amino acid residues at positions 446, 447, 479, 483, 484, 486, 487, 490, 491, 496, 498, 499, 500, 531, 534, 537, and 538 of Fok I are all targets for influencing dimerization of the Fok I cleavage half-domains. Exemplary engineered cleavage monomers of Fok I that form obligate heterodimers include a pair in which a first cleavage monomer includes mutations at amino acid residue positions 490 and 538 of Fok I and a second cleavage monomer that includes mutations at amino-acid residue positions 486 and 499.

Thus, in one embodiment, a mutation at amino acid position 490 replaces Glu (E) with Lys (K); a mutation at amino acid residue 538 replaces Iso (I) with Lys (K); a mutation at amino acid residue 486 replaces Gln (Q) with Glu (E); and a mutation at position 499 replaces Iso (I) with Lys (K). Specifically, the engineered cleavage monomers may be prepared by mutating positions 490 from E to K and 538 from I to K in one cleavage monomer to produce an engineered cleavage monomer designated “E490K:1538K” and by mutating positions 486 from Q to E and 499 from I to L in another cleavage monomer to produce an engineered cleavage monomer designated “Q486E:I499L.” The above described engineered cleavage monomers are obligate heterodimer mutants in which aberrant cleavage is minimized or abolished. Engineered cleavage monomers may be prepared using a suitable method, for example, by site-directed mutagenesis of wild-type cleavage monomers (Fok I) as described in U.S. Patent Publication No. 20050064474 (see Example 5).

The zinc finger nuclease described above may be engineered to introduce a double stranded break at the targeted site of integration. The double stranded break may be at the targeted site of integration, or it may be up to 1, 2, 3, 4, 5, 10, 15, 20, 25, 30, 35, 40, 45, 50, 100, or 1000 nucleotides away from the site of integration. In some embodiments, the double stranded break may be up to 1, 2, 3, 4, 5, 10, 15, or 20 nucleotides away from the site of integration. In other embodiments, the double stranded break may be up to 10, 15, 20, 25, 30, 35, 40, 45, or 50 nucleotides away from the site of integration. In yet other embodiments, the double stranded break may be up to 50, 100, or 1000 nucleotides away from the site of integration.

### (b) Optional Donor Polynucleotide

The method for editing chromosomal sequences involved in tumor suppression may further comprise introducing at least one donor polynucleotide comprising a sequence encoding a protein involved in tumor suppression into the embryo or cell. A donor polynucleotide comprises at least three components: the sequence coding the protein involved in tumor suppression, an upstream sequence, and a downstream sequence. The sequence encoding the protein involved in tumor suppression is flanked by the upstream and downstream sequence, wherein the upstream and downstream sequences share sequence similarity with either side of the site of integration in the chromosome.

Typically, the donor polynucleotide will be DNA. The donor polynucleotide may be a DNA plasmid, a bacterial artificial chromosome (BAC), a yeast artificial chromosome (YAC), a viral vector, a linear piece of DNA, a PCR fragment, a naked nucleic acid, or a nucleic acid complexed with a delivery vehicle such as a liposome or poloxamer. An exemplary donor polynucleotide comprising the sequence encoding a protein involved in tumor suppression may be a BAC.

The sequence of the donor polynucleotide that encodes the protein involved in tumor suppression may include coding (i.e., exon) sequence, as well as intron sequences and upstream regulatory sequences (such as, e.g., a promoter). Depending upon the identity and the source of the orthologous protein involved in tumor suppression, the size of the sequence encoding the protein involved in tumor suppression can and will vary. For example, the sequence encoding the protein involved in tumor suppression may range in size from about 1 kb to about 5,000 kb.

The donor polynucleotide also comprises upstream and downstream sequences flanking the chromosomal sequence involved in tumor suppression. The upstream and downstream sequences in the donor polynucleotide are selected to promote recombination between the chromosomal sequence of interest and the donor polynucleotide. The upstream sequence, as used herein, refers to a nucleic acid sequence that shares sequence similarity with the chromosomal sequence upstream of the targeted site of integration. Similarly, the downstream sequence refers to a nucleic acid sequence that shares sequence similarity with the chromosomal sequence downstream of the targeted site of integration. The upstream and downstream sequences in the donor polynucleotide may share about 75%, 80%, 85%, 90%, 95%, or 100% sequence identity with the targeted chromosomal sequence. In other embodiments, the upstream and downstream sequences in the donor polynucleotide may share about 95%, 96%, 97%, 98%, 99%, or 100% sequence identity with the targeted chromosomal sequence. In an exemplary embodiment, the upstream and downstream sequences in the donor polynucleotide may share about 99% or 100% sequence identity with the targeted chromosomal sequence.

An upstream or downstream sequence may comprise from about 50 bp to about 2500 bp. In one embodiment, an upstream or downstream sequence may comprise about 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. An exemplary upstream or downstream sequence may comprise about 200 bp to about 2000 bp, about 600 bp to about 1000 bp, or more particularly about 700 bp to about 1000 bp.

In some embodiments, the donor polynucleotide may further comprise a marker. Such a marker may make it easy to screen for targeted integrations. Non-limiting examples of suitable markers include restriction sites, fluorescent proteins, or selectable markers.

One of skill in the art would be able to construct a donor polynucleotide as described herein using well-known standard recombinant techniques (see, for example, Sambrook et al., 2001 and Ausubel et al., 1996).

In the method detailed above for integrating a chromosomal sequence involved in tumor suppression, a double stranded break introduced into the chromosomal sequence by the zinc finger nuclease is repaired, via homologous recombination with the donor polynucleotide, such that the chromosomal sequence involved in tumor suppression is integrated into the chromosome. The presence of a double-stranded break facilitates integration of the sequence into the chromosome. A donor polynucleotide may be physically integrated or, alternatively, the donor polynucleotide may be used as a template for repair of the break, resulting in the introduction of the chromosomal sequence involved in tumor suppression as well as all or part of the upstream and downstream sequences of the donor polynucleotide into the chromosome. Thus, endogenous chromosomal sequence may be converted to the sequence of the donor polynucleotide.

### (c) Optional Exchange Polynucleotide

The method for editing chromosomal sequences involved in tumor suppression may further comprise introducing into the embryo or cell at least one exchange polynucleotide comprising a sequence that is substantially identical to the chromosomal sequence at the site of cleavage and which further comprises at least one specific nucleotide change.

Typically, the exchange polynucleotide will be DNA. The exchange polynucleotide may be a DNA plasmid, a bacterial artificial chromosome (BAC), a yeast artificial chromosome (YAC), a viral vector, a linear piece of DNA, a PCR fragment, a naked nucleic acid, or a nucleic acid complexed with a delivery vehicle such as a liposome or poloxamer. An exemplary exchange polynucleotide may be a DNA plasmid.

The sequence in the exchange polynucleotide is substantially identical to a portion of the chromosomal sequence at the site of cleavage. In general, the sequence of the exchange polynucleotide will share enough sequence identity with the chromosomal sequence such that the two sequences may be exchanged by homologous recombination. For example, the sequence in the exchange polynucleotide may have at least about 80, 81, 82, 83, 84, 85, 86, 87, 88, 89, 90, 91, 92, 93, 94, 95, 96, 97, 98, or 99% sequence identity with a portion of the chromosomal sequence.

Importantly, the sequence in the exchange polynucleotide comprises at least one specific nucleotide change with respect to the sequence of the corresponding chromosomal sequence. For example, one nucleotide in a specific codon may be changed to another nucleotide such that the codon codes for a different amino acid. In one embodiment, the sequence in the exchange polynucleotide may comprise one specific nucleotide change such that the encoded protein comprises one amino acid change. In other embodiments, the sequence in the exchange polynucleotide may comprise two, three, four, or more specific nucleotide changes such that the encoded protein comprises one, two, three, four, or more amino acid changes. In still other embodiments, the sequence in the exchange polynucleotide may comprise a three nucleotide deletion or insertion such that the reading frame of the coding reading is not altered (and a functional protein is produced). The expressed protein, however, would comprise a single amino acid deletion or insertion.

The length of the sequence in the exchange polynucleotide that is substantially identical to a portion of the chromosomal sequence at the site of cleavage can and will vary. In general, the sequence in the exchange polynucleotide may range from about 50 bp to about 10,000 bp in length. In various embodiments, the sequence in the exchange polynucleotide may be about 100, 200, 400, 600, 800, 1000, 1200, 1400, 1600, 1800, 2000, 2200, 2400, 2600, 2800, 3000, 3200, 3400, 3600, 3800, 4000, 4200, 4400, 4600, 4800, or 5000 bp in length. In other embodiments, the sequence in the exchange polynucleotide may be about 5500, 6000, 6500, 6000, 6500, 7000, 7500, 8000, 8500, 9000, 9500, or 10,000 bp in length.

One of skill in the art would be able to construct an exchange polynucleotide as described herein using well-known standard recombinant techniques (see, for example, Sambrook et al., 2001 and Ausubel et al., 1996).

In the method detailed above for modifying a chromosomal sequence, a double stranded break introduced into the chromosomal sequence by the zinc finger nuclease is repaired, via homologous recombination with the exchange polynucleotide, such that the sequence in the exchange polynucleotide may be exchanged with a portion of the chromosomal sequence. The presence of the double stranded break facilitates homologous recombination and repair of the break. The exchange polynucleotide may be physically integrated or, alternatively, the exchange polynucleotide may be used as a template for repair of the break, resulting in the exchange of the sequence information in the exchange polynucleotide with the sequence information in that portion of the chromosomal sequence. Thus, a portion of the endogenous chromosomal sequence may be converted to the sequence of the exchange polynucleotide. The changed nucleotide(s) may be at or near the site of cleavage. Alternatively, the changed nucleotide(s) may be anywhere in the exchanged sequences. As a consequence of the exchange, however, the chromosomal sequence is modified.

### (d) Delivery of Nucleic Acids

To mediate zinc finger nuclease genomic editing, at least one nucleic acid molecule encoding a zinc finger nuclease and, optionally, at least one exchange polynucleotide or at least one donor polynucleotide are delivered to the embryo or the cell of interest. Typically, the embryo is a fertilized one-cell stage embryo of the species of interest.

Suitable methods of introducing the nucleic acids to the embryo or cell include microinjection, electroporation, sonoporation, biolistics, calcium phosphate-mediated transfection, cationic transfection, liposome transfection, dendrimer transfection, heat shock transfection, nucleofection transfection, magnetofection, lipofection, impalefection, optical transfection, proprietary agent-enhanced uptake of nucleic acids, and delivery via liposomes, immunoliposomes, virosomes, or artificial virions. In one embodiment, the nucleic acids may be introduced into an embryo by microinjection. The nucleic acids may be microinjected into the nucleus or the cytoplasm of the embryo. In another embodiment, the nucleic acids may be introduced into a cell by nucleofection.

In embodiments in which both a nucleic acid encoding a zinc finger nuclease and a donor (or exchange) polynucleotide are introduced into an embryo or cell, the ratio of donor (or exchange) polynucleotide to nucleic acid encoding a zinc finger nuclease may range from about 1:10 to about 10:1. In various embodiments, the ratio of donor (or exchange) polynucleotide to nucleic acid encoding a zinc finger nuclease may be about 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, or 10:1. In one embodiment, the ratio may be about 1:1.

In embodiments in which more than one nucleic acid encoding a zinc finger nuclease and, optionally, more than one donor (or exchange) polynucleotide are introduced into an embryo or cell, the nucleic acids may be introduced simultaneously or sequentially. For example, nucleic acids encoding the zinc finger nucleases, each specific for a distinct recognition sequence, as well as the optional donor (or exchange) polynucleotides, may be introduced at the same time. Alternatively, each nucleic acid encoding a zinc finger nuclease, as well as the optional donor (or exchange) polynucleotides, may be introduced sequentially.

In some embodiments, the embryo may be derived from an animal comprising at least one edited chromosomal sequence involved in tumor suppression. Thus, an animal comprising 2, 3, 4, or more edited chromosomal sequences involved in tumor suppression may be generated.

### (e) Culturing the Embryo or Cell

The method of inducing genomic editing with a zinc finger nuclease further comprises culturing the embryo or cell comprising the introduced nucleic acid(s) to allow expression of the zinc finger nuclease. An embryo may be cultured in vitro (e.g., in cell culture). Typically, the embryo is cultured at an appropriate temperature and in appropriate media with the necessary O₂/CO₂ ratio to allow the expression of the zinc finger nuclease. Suitable non-limiting examples of media include M2, M16, KSOM, BMOC, and HTF media. A skilled artisan will appreciate that culture conditions can and will vary depending on the species of embryo. Routine optimization may be used, in all cases, to determine the best culture conditions for a particular species of embryo. In some cases, a cell line may be derived from an in vitro-cultured embryo (e.g., an embryonic stem cell line).

Alternatively, an embryo may be cultured in vivo by transferring the embryo into the uterus of a female host. Generally speaking, the female host is from the same or similar species as the embryo. Preferably, the female host is pseudo-pregnant. Methods of preparing pseudo-pregnant female hosts are known in the art. Additionally, methods of transferring an embryo into a female host are known. Culturing an embryo in vivo permits the embryo to develop and may result in a live birth of an animal derived from the embryo. Such an animal would comprise the edited chromosomal sequence involved in tumor suppression in every cell of the body.

Similarly, cells comprising the introduced nucleic acids may be cultured using standard procedures to allow expression of the zinc finger nuclease. Standard cell culture techniques are described, for example, in Santiago et al. (2008) PNAS 105:5809-5814; Moehle et al. (2007) PNAS 104:3055-3060; Urnov et al. (2005) Nature 435:646-651; and Lombardo et al (2007) Nat. Biotechnology 25:1298-1306. Those of skill in the art appreciate that methods for culturing cells are known in the art and can and will vary depending on the cell type. Routine optimization may be used, in all cases, to determine the best techniques for a particular cell type.

Upon expression of the zinc finger nuclease, the chromosomal sequence may be edited. In cases in which the embryo or cell comprises an expressed zinc finger nuclease but no donor (or exchange) polynucleotide, the zinc finger nuclease recognizes, binds, and cleaves the target sequence in the chromosomal sequence of interest. The double-stranded break introduced by the zinc finger nuclease is repaired by an error-prone non-homologous end-joining DNA repair process. Consequently, a deletion, insertion, or point mutation may be introduced in the chromosomal sequence such that the sequence is inactivated.

In cases in which the embryo or cell comprises an expressed zinc finger nuclease as well as a donor (or exchange) polynucleotide, the zinc finger nuclease recognizes, binds, and cleaves the target sequence in the chromosome. The double-stranded break introduced by the zinc finger nuclease is repaired, via homologous recombination with the donor (or exchange) polynucleotide, such that the sequence in the donor polynucleotide is integrated into the chromosomal sequence (or a portion of the chromosomal sequence is converted to the sequence in the exchange polynucleotide). As a consequence, a sequence may be integrated into the chromosomal sequence (or a portion of the chromosomal sequence may be modified).

The genetically modified animals disclosed herein may be crossbred to create animals comprising more than one edited chromosomal sequence or to create animals that are homozygous for one or more edited chromosomal sequences. For example, two animals comprising the same edited chromosomal sequence may be crossbred to create an animal homozygous for the edited chromosomal sequence. Alternatively, animals with different edited chromosomal sequences may be crossbred to create an animal comprising both edited chromosomal sequences.

For example, animal A comprising an inactivated ATM chromosomal sequence may be crossed with animal B comprising a chromosomally integrated sequence encoding a human ATM protein to give rise to a “humanized” ATM 1 offspring comprising both the inactivated ATM chromosomal sequence and the chromosomally integrated human ATM sequence. Similarly, an animal comprising an inactivated Notch 1 chromosomal sequence may be crossed with an animal comprising a chromosomally integrated sequence encoding the human Notch 1 protein to generate “humanized” Notch 1 offspring. Moreover, a humanized ATM animal may be crossed with a humanized Notch 1 animal to create a humanized ATM/Notch 1 animal. Those of skill in the art will appreciate that many combinations are possible.

In other embodiments, an animal comprising an edited chromosomal sequence disclosed herein may be crossbred to combine the edited chromosomal sequence with other genetic backgrounds. By way of non-limiting example, other genetic backgrounds may include wild-type genetic backgrounds, genetic backgrounds with deletion mutations, genetic backgrounds with other targeted integrations, and genetic backgrounds with non-targeted integrations.

### (IV) Applications

A further aspect of the present disclosure encompasses methods for using the genetically modified animals. In one embodiment, a genetically modified animal comprising an inactivated chromosomal sequence involved with tumor suppression may be used to determine susceptibility to developing tumors. The method comprises exposing the genetically modified animal comprising an inactivated tumor suppressor sequence and a wild-type animal to a carcinogenic agent, and then monitoring the development of tumors. The animal comprising the inactivated tumor suppressor sequence may have an increased risk for tumor formation. Moreover, an animal homozygous for the inactivated tumor suppressor sequence may have increased risk relative to an animal heterozygous for the same inactivated sequence, which in turn may have increased risk relative to a wild-type animal. A similar method may be used to screen for spontaneous tumors, wherein the animals are not exposed to a carcinogenic agent.

In another embodiment, an animal comprising an inactivated chromosomal sequence involved with tumor suppression may be used to evaluate the carcinogenic potential of a test agent. The method comprises contacting the genetically modified animal comprising an inactivated tumor suppressor sequence and a wild-type animal to the test agent, and then monitoring the development of tumors. If the animal comprising an inactivated tumor suppressor sequence has an increased incidence of tumors relative to the wild-type animal, the test agent may be carcinogenic.

In a further embodiment, an animal comprising an inactivated chromosomal sequence involved with tumor suppression may be used to determine the efficacy and/or toxicity of a chemotherapeutic agent or a combination of chemotherapeutic agents. The method comprises inducing tumor formation in genetically modified animal comprising inactivated tumor suppressor sequences, and then comparing the responses of a first group of animals contacted with the chemotherapeutic agent or combination of chemotherapeutic agents to a second group of animal not contacted with the chemotherapeutic agent or combination of chemotherapeutic agents.

In still another embodiment, an animal comprising an inactivated chromosomal sequence involved with tumor suppression may be used to screen libraries of small molecule drugs for potentially advantageous effects, including enhanced potency as well as reduced untoward effects. The method comprises inducing tumor formation in genetically modified animal comprising inactivated tumor suppressor sequences, and then comparing the responses of a first group of animals contacted with the small molecule drug candidate to a second group of animal not contacted with the small molecule drug candidate.

In another embodiment, a genetically modified animal comprising an inactivated chromosomal sequence involved with tumor suppression may be used test the ADME/Tox profile of a chemotherapeutic agent or a combination of chemotherapeutic agents. The method is similar to those detailed above, and assessment parameters include damage to DNA, metabolic consequence, and behavioral effects of the chemotherapeutic agent or the combination of chemotherapeutic agents. Behavioral tests include test of learning/memory, anxiety/depression, and sensori-motor functions. Non-limiting examples of behavioral tests suitable for assessing the motor function of rats includes open field locomoter activity assessment, the rotarod test, the grip strength test, the cylinder test, the limb-placement or grid walk test, the vertical pole test, the Inverted grid test, the adhesive removal test, the painted paw or catwalk (gait) tests, the beam traversal test, and the inclined plane test. Non-limiting examples of behavioral tests suitable for assessing the long-term memory function of rats include the elevated plus maze test, the Morris water maze swim test, contextual fear conditioning, the Y-maze test, the T-maze test, the novel object recognition test, the active avoidance test, the passive (inhibitory) avoidance test, the radial arm maze test, the two-choice swim test, the hole board test, the olfactory discrimination (go-no-go) test, and the pre-pulse inhibition test. Non-limiting examples of behavioral tests suitable for assessing the anxiety of rats include the open field locomotion assessment, observations of marble-burying behavior, the elevated plus maze test, the light/dark box test. Non-limiting examples of behavioral tests suitable for assessing the depression of rats includes the forced swim test, the tail suspension test, the hot plate test, the tail suspension test, anhedonia observations, and the novelty suppressed feeding test.

In yet another embodiment, the genetically modified animals disclosed herein may be used for gene therapy. For example, an animal having a natural mutation in a tumor suppressor gene may genetically modified by editing the chromosomal sequence comprising the natural mutation such that the mutation is corrected. Accordingly, the animal may no longer be susceptible to tumor formation or cancer development.

Still yet another aspect encompasses a method of generating a cell line or cell lysate using a genetically modified animal comprising an edited chromosomal sequence involved in tumor suppression. An additional other aspect encompasses a method of producing purified biological components using a genetically modified cell or animal comprising an edited chromosomal sequence involved in tumor suppression. Non-limiting examples of biological components include antibodies, receptor proteins, altered tumor suppressor proteins, and the like.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

The term “chromosomal sequence involved in tumor suppression” refers to a chromosomal sequence which has been identified to contribute to cell cycle maintenance, division of cells, and/or the cell death cycle. Any chromosomal sequence thought to be involved in tumor suppression will work for purposes of the present invention. Exemplary chromosomal sequences involved in tumor suppression include, but are not limited to, ATM (ataxia telangiectasia mutated), ATR (ataxia telangiectasia and Rad3 related), EGFR (epidermal growth factor receptor), ERBB2 (v-erb-b2 erythroblastic leukemia viral oncogene homolog 2), ERBB3 (v-erb-b2 erythroblastic leukemia viral oncogene homolog 3), ERBB4 (v-erb-b2 erythroblastic leukemia viral oncogene homolog 4), Notch 1, Notch 2, Notch 3, Notch 4, ATK1 (v-akt murine thymoma viral oncogene homolog 1), ATK2 (v-akt murine thymoma viral oncogene homolog 2), ATK3 (v-akt murine thymoma viral oncogene homolog 3), HIF1a (hypoxia-inducible factor 1a), HIF3a (hypoxia-inducible factor 1a), Met (met pronto-oncogene), HRG (histidine-rich glycoprotein), Bc12, PPAR(alpha) (peroxisome proliferator-activated receptor alpha), Ppar(gamma) (peroxisome proliferator-activated receptor gamma), WT1 (Wilmus Tumor 1), FGF1R (fibroblast growth factor 1 receptor), FGF2R (fibroblast growth factor 1 receptor), FGF3R (fibroblast growth factor 3 receptor), FGF4R (fibroblast growth factor 4 receptor), FGF5R (fibroblast growth factor 5 receptor), CDKN2a (cyclin-dependent kinase inhibitor 2A), APC (adenomatous polyposis coli), Rb1 (retinoblastoma 1), MEN1 (multiple endocrine neoplasia1), VHL (von-Hippel-Lindau tumor suppressor), BRCA1 (breast cancer 1), BRCA2 (breast cancer 2), AR (androgen receptor), TSG101(tumor susceptibility gene 101), Igf1 (insulin-like growth factor 1), Igf2 (insulin-like growth factor 2), Igf 1R (insulin-like growth factor 1 receptor), Igf 2R (insulin-like growth factor 2 receptor), Bax (BCL-2 associated X protein), CASP 1 (Caspase 1), CASP 2 (Caspase 2), CASP 3 (Caspase 3), CASP 4(Caspase 4), CASP 6 (Caspase 6), CASP 7 (Caspase 7), CASP 8 (Caspase 8), CASP 9 (Caspase 9), CASP 12 (Caspase 12), Kras (v-Ki-ras2 Kirsten rate sarcoma viral oncogene homolog), PTEN (phosphate and tensin homolog), BCRP (breast cancer receptor protein), p53, and combinations thereof.

The term “a protein encoded by a chromosomal sequence involved in tumor suppression” or “a protein involved in tumor suppression” refers to a protein that has been encoded by a chromosomal sequence which has been identified to contribute to cell cycle maintenance, division of cells, and/or the cell death cycle. Any chromosomal sequence thought to be involved in tumor suppression will work for purposes of the present invention. Exemplary chromosomal sequences involved in tumor suppression include, but are not limited to, ATM (ataxia telangiectasia mutated), ATR (ataxia telangiectasia and Rad3 related), EGFR (epidermal growth factor receptor), ERBB2 (v-erb-b2 erythroblastic leukemia viral oncogene homolog 2), ERBB3 (v-erb-b2 erythroblastic leukemia viral oncogene homolog 3), ERBB4 (v-erb-b2 erythroblastic leukemia viral oncogene homolog 4), Notch 1, Notch 2, Notch 3, Notch 4, ATK1 (v-akt murine thymoma viral oncogene homolog 1), ATK2 (v-akt murine thymoma viral oncogene homolog 2), ATK3 (v-akt murine thymoma viral oncogene homolog 3), HIF1a (hypoxia-inducible factor 1a), HIF3a (hypoxia-inducible factor 1a), Met (met pronto-oncogene), HRG (histidine-rich glycoprotein), Bc12, PPAR(alpha) (peroxisome proliferator-activated receptor alpha), Ppar(gamma) (peroxisome proliferator-activated receptor gamma), WT1 (Wilmus Tumor 1), FGF1R (fibroblast growth factor 1 receptor), FGF2R (fibroblast growth factor 1 receptor), FGF3R (fibroblast growth factor 3 receptor), FGF4R (fibroblast growth factor 4 receptor), FGF5R (fibroblast growth factor 5 receptor), CDKN2a (cyclin-dependent kinase inhibitor 2A), APC (adenomatous polyposis coli), Rb1 (retinoblastoma 1), MEN1 (multiple endocrine neoplasia1), VHL (von-Hippel-Lindau tumor suppressor), BRCA1 (breast cancer 1), BRCA2 (breast cancer 2), AR (androgen receptor), TSG101(tumor susceptibility gene 101), Igf1 (insulin-like growth factor 1), Igf2 (insulin-like growth factor 2), Igf 1R (insulin-like growth factor 1 receptor), Igf 2R (insulin-like growth factor 2 receptor), Bax (BCL-2 associated X protein), CASP 1 (Caspase 1), CASP 2 (Caspase 2), CASP 3 (Caspase 3), CASP 4(Caspase 4), CASP 6 (Caspase 6), CASP 7(Caspase 7), CASP 8 (Caspase 8), CASP 9 (Caspase 9), CASP 12 (Caspase 12), Kras (v-Ki-ras2 Kirsten rate sarcoma viral oncogene homolog), PTEN (phosphate and tensin homolog), BCRP (breast cancer receptor protein), P53, and combinations thereof. Any type of protein involved in tumor suppression will work for purposes of the present invention including, but not limited to, structural proteins, enzyme and catalytic proteins, transport proteins, hormonal proteins, contractile proteins, storage proteins, genetic proteins, defense proteins, and receptor proteins.

A “gene,” as used herein, refers to a DNA region (including exons and introns) encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions.

The terms “nucleic acid” and “polynucleotide” refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogs of natural nucleotides, as well as nucleotides that are modified in the base, sugar and/or phosphate moieties (e.g., phosphorothioate backbones). In general, an analog of a particular nucleotide has the same base-pairing specificity; i.e., an analog of A will base-pair with T.

The terms “polypeptide” and “protein” are used interchangeably to refer to a polymer of amino acid residues.

The term “recombination” refers to a process of exchange of genetic information between two polynucleotides. For the purposes of this disclosure, “homologous recombination” refers to the specialized form of such exchange that takes place, for example, during repair of double-strand breaks in cells. This process requires sequence similarity between the two polynucleotides, uses a “donor” or “exchange” molecule to template repair of a “target” molecule (i.e., the one that experienced the double-strand break), and is variously known as “non-crossover gene conversion” or “short tract gene conversion,” because it leads to the transfer of genetic information from the donor to the target. Without being bound by any particular theory, such transfer can involve mismatch correction of heteroduplex DNA that forms between the broken target and the donor, and/or “synthesis-dependent strand annealing,” in which the donor is used to resynthesize genetic information that will become part of the target, and/or related processes. Such specialized homologous recombination often results in an alteration of the sequence of the target molecule such that part or all of the sequence of the donor polynucleotide is incorporated into the target polynucleotide.

As used herein, the terms “target site” or “target sequence” refer to a nucleic acid sequence that defines a portion of a chromosomal sequence to be edited and to which a zinc finger nuclease is engineered to recognize and bind, provided sufficient conditions for binding exist.

Techniques for determining nucleic acid and amino acid sequence identity are known in the art. Typically, such techniques include determining the nucleotide sequence of the mRNA for a gene and/or determining the amino acid sequence encoded thereby, and comparing these sequences to a second nucleotide or amino acid sequence. Genomic sequences can also be determined and compared in this fashion. In general, identity refers to an exact nucleotide-to-nucleotide or amino acid-to-amino acid correspondence of two polynucleotides or polypeptide sequences, respectively. Two or more sequences (polynucleotide or amino acid) can be compared by determining their percent identity. The percent identity of two sequences, whether nucleic acid or amino acid sequences, is the number of exact matches between two aligned sequences divided by the length of the shorter sequences and multiplied by 100. An approximate alignment for nucleic acid sequences is provided by the local homology algorithm of Smith and Waterman, Advances in Applied Mathematics 2:482-489 (1981). This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986). An exemplary implementation of this algorithm to determine percent identity of a sequence is provided by the Genetics Computer Group (Madison, Wis.) in the “BestFit” utility application. Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found on the GenBank website. With respect to sequences described herein, the range of desired degrees of sequence identity is approximately 80% to 100% and any integer value therebetween. Typically the percent identities between sequences are at least 70-75%, preferably 80-82%, more preferably 85-90%, even more preferably 92%, still more preferably 95%, and most preferably 98% sequence identity.

Alternatively, the degree of sequence similarity between polynucleotides can be determined by hybridization of polynucleotides under conditions that allow formation of stable duplexes between regions that share a degree of sequence identity, followed by digestion with single-stranded-specific nuclease(s), and size determination of the digested fragments. Two nucleic acid, or two polypeptide sequences are substantially similar to each other when the sequences exhibit at least about 70%-75%, preferably 80%-82%, more-preferably 85%-90%, even more preferably 92%, still more preferably 95%, and most preferably 98% sequence identity over a defined length of the molecules, as determined using the methods above. As used herein, substantially similar also refers to sequences showing complete identity to a specified DNA or polypeptide sequence. DNA sequences that are substantially similar can be identified in a Southern hybridization experiment under, for example, stringent conditions, as defined for that particular system. Defining appropriate hybridization conditions is within the skill of the art. See, e.g., Sambrook et al., supra; Nucleic Acid Hybridization: A Practical Approach, editors B. D. Hames and S. J. Higgins, (1985) Oxford; Washington, D.C.; IRL Press).

Selective hybridization of two nucleic acid fragments can be determined as follows. The degree of sequence identity between two nucleic acid molecules affects the efficiency and strength of hybridization events between such molecules. A partially identical nucleic acid sequence will at least partially inhibit the hybridization of a completely identical sequence to a target molecule. Inhibition of hybridization of the completely identical sequence can be assessed using hybridization assays that are well known in the art (e.g., Southern (DNA) blot, Northern (RNA) blot, solution hybridization, or the like, see Sambrook, et al., Molecular Cloning: A Laboratory Manual, Second Edition, (1989) Cold Spring Harbor, N.Y.). Such assays can be conducted using varying degrees of selectivity, for example, using conditions varying from low to high stringency. If conditions of low stringency are employed, the absence of non-specific binding can be assessed using a secondary probe that lacks even a partial degree of sequence identity (for example, a probe having less than about 30% sequence identity with the target molecule), such that, in the absence of non-specific binding events, the secondary probe will not hybridize to the target.

When utilizing a hybridization-based detection system, a nucleic acid probe is chosen that is complementary to a reference nucleic acid sequence, and then by selection of appropriate conditions the probe and the reference sequence selectively hybridize, or bind, to each other to form a duplex molecule. A nucleic acid molecule that is capable of hybridizing selectively to a reference sequence under moderately stringent hybridization conditions typically hybridizes under conditions that allow detection of a target nucleic acid sequence of at least about 10-14 nucleotides in length having at least approximately 70% sequence identity with the sequence of the selected nucleic acid probe. Stringent hybridization conditions typically allow detection of target nucleic acid sequences of at least about 10-14 nucleotides in length having a sequence identity of greater than about 90-95% with the sequence of the selected nucleic acid probe. Hybridization conditions useful for probe/reference sequence hybridization, where the probe and reference sequence have a specific degree of sequence identity, can be determined as is known in the art (see, for example, Nucleic Acid Hybridization: A Practical Approach, editors B. D. Hames and S. J. Higgins, (1985) Oxford; Washington, D.C.; IRL Press). Conditions for hybridization are well-known to those of skill in the art.

Hybridization stringency refers to the degree to which hybridization conditions disfavor the formation of hybrids containing mismatched nucleotides, with higher stringency correlated with a lower tolerance for mismatched hybrids. Factors that affect the stringency of hybridization are well-known to those of skill in the art and include, but are not limited to, temperature, pH, ionic strength, and concentration of organic solvents such as, for example, formamide and dimethylsulfoxide. As is known to those of skill in the art, hybridization stringency is increased by higher temperatures, lower ionic strength and lower solvent concentrations. With respect to stringency conditions for hybridization, it is well known in the art that numerous equivalent conditions can be employed to establish a particular stringency by varying, for example, the following factors: the length and nature of the sequences, base composition of the various sequences, concentrations of salts and other hybridization solution components, the presence or absence of blocking agents in the hybridization solutions (e.g., dextran sulfate, and polyethylene glycol), hybridization reaction temperature and time parameters, as well as, varying wash conditions. A particular set of hybridization conditions may be selected following standard methods in the art (see, for example, Sambrook, et al., Molecular Cloning: A Laboratory Manual, Second Edition, (1989) Cold Spring Harbor, N.Y.).

### Examples

The following examples are included to illustrate the invention.

### Example 1

### Identification of ZFNs that Edit the p53 Locus

The p53 gene was chosen for zinc finger nuclease (ZFN) mediated genome editing. ZFNs were designed, assembled, and validated using strategies and procedures previously described (see Geurts et al. Science (2009) 325:433). ZFN design made use of an archive of pre-validated 1-finger and 2-finger modules. The rat p53 gene region (NM_{—}030989) was scanned for putative zinc finger binding sites to which existing modules could be fused to generate a pair of 4-, 5-, or 6-finger proteins that would bind a 12-18 bp sequence on one strand and a 12-18 bp sequence on the other strand, with about 5-6 bp between the two binding sites.

Capped, polyadenylated mRNA encoding each pair of ZFNs was produced using known molecular biology techniques. The mRNA was transfected into rat cells. Control cells were transfected with mRNA encoding GFP. Active ZFN pairs were identified by detecting ZFN-induced double strand chromosomal breaks using the Cel-1 nuclease assay. This assay detects alleles of the target locus that deviate from wild type as a result of non-homologous end joining (NHEJ)-mediated imperfect repair of ZFN-induced DNA double strand breaks. PCR amplification of the targeted region from a pool of ZFN-treated cells generates a mixture of WT and mutant amplicons. Melting and reannealing of this mixture results in mismatches forming between heteroduplexes of the WT and mutant alleles. A DNA “bubble” formed at the site of mismatch is cleaved by the surveyor nuclease Cel-1, and the cleavage products can be resolved by gel electrophoresis. This assay revealed that the ZFN pair targeted to bind 5′-atCTGGAGGAAGACtGGAGAAcaagagc-3′ (SEQ ID NO:3; contact sites shown in uppercase) and 5′-atATTCTGGTAAGGAGCCGGgcaagagg-3′ (SEQ ID NO:4) edited the p53 gene.

### Example 2

### Editing of the p53 Locus in Rat Embryos

Capped, polyadenylated mRNA encoding the active pair of ZFNs was microinjected into fertilized rat embryos using standard procedures (e.g., see Geurts et al. (2009) supra). Control embryos were microinjected with saline or mRNA encoding GFP. The injected embryos were transferred to pseudopregnant female rats to be carried to parturition. Toe/tail of clips of each live born animal was harvested for DNA extraction and analysis using a Cel-1 assay. As shown in FIG. 1, about 25% of the experimental animals had an edited p53 gene locus.

### Example 3

### Inactivation of the p53 Locus in Rat

To determine that the edited p53 locus was inactivated, Western analyses were performed to confirm that no p53 protein was produced. Cell lysates were prepared from the kidney and liver of a wildtype animal and a p53 knockout animal. As shown on FIG. 2, both cytoplasmic and nuclear lysates of the p53 knockout animal were devoid of p53 protein. The levels of actin protein were constant among the wildtype and mutant samples, however. Thus, the p53 edited rat was a p53 knock-out rat.

### Example 4

### Identification of ZFNs that Edit the BCRP Locus in Rat

ZFNs that target and cleave the BCRP gene were identified essentially as described above in Example 1. The rat BCRP gene (NM_{—}1811381) was scanned for putative zinc finger binding sites. ZFNs were assembled and tested essentially as described in Example 1. It was found that the ZFN pair targeted to bind 5′-atGACGTCAAGGAAGAAgtctgcagggt-3′ (SEQ ID NO:5) and 5′-acGGAGATTCTTCGGCTgtaatgttaaa-3′ (SEQ ID NO:6) edited the BCRP gene.

### Example 5

### Editing the BCRP Locus

Rat embryos were microinjected with mRNA encoding the active pair of BCRP ZFNs essentially as described in Examples 1 and 2. The injected embryos were incubated and DNA was extracted from the resultant animals. The targeted region of the BCRP gene was PCR amplified using appropriate primers. The amplified DNA was subcloned into a suitable vector and sequenced using standard methods. FIG. 3 presents edited BCRP loci in two founder animals. One animal had a 588 bp deletion in exon 7, and the second animal had a 696 bp deletion in exon 7. These deletions disrupt the reading frame of the BCRP coding region.

### Example 6

### Editing the Pten Locus

ZFNs that target and cleave the Pten locus in rats were designed and tested for activity essentially as described above in Example 1. An active pair of ZFNs was identified. The DNA binding sites were 5′-CCCCAGTTTGTGGTCtgcca-3′ (SEQ ID NO:7) and 5′-gcTAAAGGTGAAGATCTA-3′ (SEQ ID NO:8). Capped, polyadenylated mRNA encoding the active pair may be microinjected into rat embryos and the resultant embryos may be analyzed as described in Examples 1 and 2. Accordingly, the Pten locus may be edited to contain a deletion or an insertion such that the coding region is disrupted and no functional protein is made.

The table below presents the amino acid sequences of helices of the active ZFNs.

| Name | Sequence of Zinc Finger Helices | SEQ ID NO: |
|---|---|---|
| p53 | QSGNLAR QSGHLSR DRSALSR QSGNLAR | 9 |
| | RSDALSR RSDALTQ | |
| p53 | RSDHLSE TSSDRTK RSDHLSA QSGSLTR | 10 |
| | RSDVLSE HSNARKT | |
| BRCP | QSGNLAR QSGNLAR RSDSLST DNASRIR | 11 |
| | DRSNLTR | |
| BRCP | QSSDLSR RNDDRKK RREDLIT TSSNLSR | 12 |
| | QSGHLSR | |

What is claimed is:

1. A genetically modified animal comprising at least one edited chromosomal sequence encoding a protein involved in tumor suppression.

2. The genetically modified animal of claim 1, wherein the edited chromosomal sequence is inactivated, modified, or comprises an integrated sequence.

3. The genetically modified animal of claim 1, wherein the edited chromosomal sequence is inactivated such that no function tumor suppressor protein is produced.

4. The genetically modified animal of claim 3, wherein the inactivated chromosomal sequence comprises no exogenously introduced sequence.

5. The genetically modified animal of claim 3, further comprising at least one chromosomally integrated sequence encoding a functional protein involved in tumor suppression.

6. The genetically modified animal of claim 1, further comprising a conditional knock-out system for conditional expression of the tumor suppressor protein.

7. The genetically modified animal of claim 1, wherein the edited chromosomal sequence comprises an integrated reporter sequence.

8. The genetically modified animal of claim 1, wherein the protein involved in tumor suppression is chosen from ATM (ataxia telangiectasia mutated), ATR (ataxia telangiectasia and Rad3 related), EGFR (epidermal growth factor receptor), ERBB2 (v-erb-b2 erythroblastic leukemia viral oncogene homolog 2), ERBB3 (v-erb-b2 erythroblastic leukemia viral oncogene homolog 3), ERBB4 (v-erb-b2 erythroblastic leukemia viral oncogene homolog 4), Notch 1, Notch 2, Notch 3, Notch 4, ATK1 (v-akt murine thymoma viral oncogene homolog 1), ATK2 (v-akt murine thymoma viral oncogene homolog 2), ATK3 (v-akt murine thymoma viral oncogene homolog 3), HIF1a (hypoxia-inducible factor 1a), HIF3a (hypoxia-inducible factor 1a), Met (met pronto-oncogene), HRG (histidine-rich glycoprotein), Bc12, PPAR(alpha) (peroxisome proliferator-activated receptor alpha), Ppar(gamma) (peroxisome proliferator-activated receptor gamma), WT1 (Wilmus Tumor 1), FGF1R (fibroblast growth factor 1 receptor), FGF2R (fibroblast growth factor 1 receptor), FGF3R (fibroblast growth factor 3 receptor), FGF4R (fibroblast growth factor 4 receptor), FGF5R (fibroblast growth factor 5 receptor), CDKN2a (cyclin-dependent kinase inhibitor 2A), APC (adenomatous polyposis coli), Rb1 (retinoblastoma 1), MEN1 (multiple endocrine neoplasia1), VHL (von-Hippel-Lindau tumor suppressor), BRCA1 (breast cancer 1), BRCA2 (breast cancer 2), AR (androgen receptor), TSG101 (tumor susceptibility gene 101), Igf1 (insulin-like growth factor 1), Igf2 (insulin-like growth factor 2), Igf 1R (insulin-like growth factor 1 receptor), Igf 2R (insulin-like growth factor 2 receptor), Bax (BCL-2 associated X protein), CASP 1 (Caspase 1), CASP 2 (Caspase 2), CASP 3 (Caspase 3), CASP 4(Caspase 4), CASP 6 (Caspase 6), CASP 7 (Caspase 7), CASP 8 (Caspase 8), CASP 9 (Caspase 9), CASP 12 (Caspase 12), Kras (v-Ki-ras2 Kirsten rate sarcoma viral oncogene homolog), PTEN (phosphate and tensin homolog), BCRP (breast cancer receptor protein), P53, and combinations thereof.

9. The genetically modified animal of claim 1, wherein the protein involved in tumor suppression is chosen from BCRP, p53, PTEN, and combinations thereof.

10. The genetically modified animal of claim 1, wherein the animal is heterozygous or homozygous for the at least one edited chromosomal sequence.

11. The genetically modified animal of claim 1, wherein the animal is an embryo, a juvenile, or an adult.

12. The genetically modified animal of claim 1, wherein the animal is chosen from bovine, canine, equine, feline, ovine, porcine, non-human primate, and rodent.

13. The genetically modified animal of claim 5, wherein the animal is rat, and the chromosomally integrated sequence encoding a protein involved in tumor suppression is human.

14. A non-human embryo, the embryo comprising at least one RNA molecule encoding a zinc finger nuclease that recognizes a chromosomal sequence encoding a protein involved in tumor suppression, and, optionally, at least one donor polynucleotide comprising a sequence encoding a protein involved in tumor suppression.

15. The non-human embryo of claim 14, wherein the protein involved in tumor suppression is chosen from ATM (ataxia telangiectasia mutated), ATR (ataxia telangiectasia and Rad3 related), EGFR (epidermal growth factor receptor), ERBB2 (v-erb-b2 erythroblastic leukemia viral oncogene homolog 2), ERBB3 (v-erb-b2 erythroblastic leukemia viral oncogene homolog 3), ERBB4 (v-erb-b2 erythroblastic leukemia viral oncogene homolog 4), Notch 1, Notch 2, Notch 3, Notch 4, ATK1 (v-akt murine thymoma viral oncogene homolog 1), ATK2 (v-akt murine thymoma viral oncogene homolog 2), ATK3 (v-akt murine thymoma viral oncogene homolog 3), HIF1a (hypoxia-inducible factor 1a), HIF3a (hypoxia-inducible factor 1a), Met (met pronto-oncogene), HRG (histidine-rich glycoprotein), Bc12, PPAR(alpha) (peroxisome proliferator-activated receptor alpha), Ppar(gamma) (peroxisome proliferator-activated receptor gamma), WT1 (Wilmus Tumor 1), FGF1R (fibroblast growth factor 1 receptor), FGF2R (fibroblast growth factor 1 receptor), FGF3R (fibroblast growth factor 3 receptor), FGF4R (fibroblast growth factor 4 receptor), FGF5R (fibroblast growth factor 5 receptor), CDKN2a (cyclin-dependent kinase inhibitor 2A), APC (adenomatous polyposis coli), Rb1 (retinoblastoma 1), MEN1 (multiple endocrine neoplasia1), VHL (von-Hippel-Lindau tumor suppressor), BRCA1 (breast cancer 1), BRCA2 (breast cancer 2), AR (androgen receptor), TSG101(tumor susceptibility gene 101), Igf1 (insulin-like growth factor 1), Igf2 (insulin-like growth factor 2), Igf 1R (insulin-like growth factor 1 receptor), Igf 2R (insulin-like growth factor 2 receptor), Bax (BCL-2 associated X protein), CASP 1 (Caspase 1), CASP 2 (Caspase 2), CASP 3 (Caspase 3), CASP 4(Caspase 4), CASP 6 (Caspase 6), CASP 7 (Caspase 7), CASP 8 (Caspase 8), CASP 9 (Caspase 9), CASP 12 (Caspase 12), Kras (v-Ki-ras2 Kirsten rate sarcoma viral oncogene homolog), PTEN (phosphate and tensin homolog), BCRP (breast cancer receptor protein), P53, and combinations thereof.

16. The non-human embryo of claim 14, wherein the embryo is chosen from bovine, canine, equine, feline, ovine, porcine, non-human primate, and rodent.

17. The non-human embryo of claim 14, wherein the embryo is rat and the donor polynucleotide comprising a sequence encoding a protein involved in tumor suppression is human.

18. A genetically modified cell, the cell comprising at least one edited chromosomal sequence encoding a protein involved in tumor suppression.

19. The genetically modified cell of claim 18, wherein the edited chromosomal sequence is inactivated, modified, or comprises an integrated sequence.

20. The genetically modified cell of claim 18, wherein the edited chromosomal sequence is inactivated such that no function tumor suppressor protein is produced.

21. The genetically modified cell of claim 20, wherein the inactivated chromosomal sequence comprises no exogenously introduced sequence.

22. The genetically modified cell of claim 20, further comprising at least one chromosomally integrated sequence encoding a protein involved in tumor suppression.

23. The genetically modified cell of claim 18, further comprising a conditional knock-out system for conditional expression of the protein involved in tumor suppression.

24. The genetically modified cell of claim 18, wherein the edited chromosomal sequence comprises an integrated reporter sequence.

25. The genetically modified cell of claim 18, wherein the cell is heterozygous or homozygous for the at least one edited chromosomal sequence.

26. The genetically modified cell of claim 18, wherein the cell is of bovine, canine, equine, feline, human, ovine, porcine, non-human primate, or rodent origin.

27. The genetically modified cell of claim 22, wherein the cell is of rat origin and the chromosomally integrated sequence encoding a protein involved in tumor suppression is human.

28. The genetically modified cell of claim 18, wherein the protein involved in tumor suppression is ATM (ataxia telangiectasia mutated), ATR (ataxia telangiectasia and Rad3 related), EGFR (epidermal growth factor receptor), ERBB2 (v-erb-b2 erythroblastic leukemia viral oncogene homolog 2), ERBB3 (v-erb-b2 erythroblastic leukemia viral oncogene homolog 3), ERBB4 (v-erb-b2 erythroblastic leukemia viral oncogene homolog 4), Notch 1, Notch 2, Notch 3, Notch 4, ATK1 (v-akt murine thymoma viral oncogene homolog 1), ATK2 (v-akt murine thymoma viral oncogene homolog 2), ATK3 (v-akt murine thymoma viral oncogene homolog 3), HIF1a (hypoxia-inducible factor 1a), HIF3a (hypoxia-inducible factor 1a), Met (met pronto-oncogene), HRG (histidine-rich glycoprotein), Bc12, PPAR(alpha) (peroxisome proliferator-activated receptor alpha), Ppar(gamma) (peroxisome proliferator-activated receptor gamma), WT1 (Wilmus Tumor 1), FGF1R (fibroblast growth factor 1 receptor), FGF2R (fibroblast growth factor 1 receptor), FGF3R (fibroblast growth factor 3 receptor), FGF4R (fibroblast growth factor 4 receptor), FGF5R (fibroblast growth factor 5 receptor), CDKN2a (cyclin-dependent kinase inhibitor 2A), APC (adenomatous polyposis coli), Rb1 (retinoblastoma 1), MEN1 (multiple endocrine neoplasia1), VHL (von-Hippel-Lindau tumor suppressor), BRCA1 (breast cancer 1), BRCA2 (breast cancer 2), AR (androgen receptor), TSG101 (tumor susceptibility gene 101), Igf1 (insulin-like growth factor 1), Igf2 (insulin-like growth factor 2), Igf 1R (insulin-like growth factor 1 receptor), Igf 2R (insulin-like growth factor 2 receptor), Bax (BCL-2 associated X protein), CASP 1 (Caspase 1), CASP 2 (Caspase 2), CASP 3 (Caspase 3), CASP 4(Caspase 4), CASP 6 (Caspase 6), CASP 7(Caspase 7), CASP 8 (Caspase 8), CASP 9 (Caspase 9), CASP 12 (Caspase 12), Kras (v-Ki-ras2 Kirsten rate sarcoma viral oncogene homolog), PTEN (phosphate and tensin homolog), BCRP (breast cancer receptor protein), and P53.

29. A zinc finger nuclease, the zinc finger nuclease comprising:
a) a zinc finger DNA binding domain that binds a sequence having at least about 80% sequence identity to a sequence chosen from SEQ ID NOs:3, 4, 5, 6, 7, and 8; and
b) a cleavage domain.

30. The zinc finger nuclease of claim 29, wherein the sequence identity is at least about 85%, 90%, 95%, or 100%.

31. The zinc finger nuclease of claim 29, wherein the DNA binding domain comprises at least three zinc finger recognition regions.

32. The zinc finger nuclease of claim 29, wherein the cleavage domain is a wild-type or an engineered Fok I cleavage domain.

33. A nucleic acid sequence recognized by a zinc finger nuclease, the nucleic acid sequence having at least about 80% sequence identity to a sequence chosen from SEQ ID NOs:3, 4, 5, 6, 7, and 8.

34. A method for assessing the therapeutic effect and/or toxicity of an agent, the method comprising:
a) contacting a first genetically modified animal comprising at least one edited chromosomal sequence encoding a protein involved in tumor suppressor with the agent;
b) measuring a therapeutic and/or toxicity response in the first animal; and
c) comparing the response in (b) to results obtained from a second genetically modified animal comprising the same edited chromosomal sequence encoding a protein involved in tumor suppression, wherein the second animal is not contacted with the agent.


SEQUENCE LISTING (12 sequences)

SEQ ID NO: 1 - DNA, 900 bp, Rattus rattus
ccaggcccacgtgtggcacacaggcatatgtgcaggcaaaatacccatatacatcaaaaaaaatttttaagaaaaaatagaataattgagcctaaataagtggggagttagtagctatgaatttgaaataggcgttaggccagtttgagagaactgtaaagttgagaccagagctaaagtgtgctgtcctgtctatgttctcttgagaatgacagaagagaattagaatctctccacttagtaactgaagagacaatgatgacgtgcagatcttgaagtcagagagcacaagaacagttaatgttttcccttccttccttccttccttccttccttccttccttccttccttccttctttctttctttctggacttggatttagatctcagacagaagactagtgagcaccagagcatatcagtagtcctccgtggctcataatttatgatatacaccaatgcatgagtttctcaagtgtccaaaactcctctggaattattggagtatttgttaagcatatgagaaaaggatgtgttagtgaagggagagtgagaatggggagaacttggcgcgttctgtcttcccaagccttccctgcctcatctgaatacaccttctctattccaagaccatcagggtcgtgtgtccttgtgttgccttctcttgcaggttaccactgtgagccctacaacaaccctgcagacttcttccttgacgtcatcaacggagattcttcggctgtaatgttaaacagaggggaacaagaccatgaaggtatatgagtcttgtagattcatacgttggtgtccaattgtttgattgaagcccagtagtgtgtcttattaaagcatgcttttgtaggccagccaggactatgtagtgagatcctaccttaaaa

SEQ ID NO: 2 - DNA, 800 bp, Rattus rattus
tcttgagaatgacagaagagaattagaatctctccacttagtaactgaagagacaatgatgacgtgcagatcttgaagtcagagagcacaagaacagttaatgttttcccttccttccttccttccttccttccttccttccttccttccttccttctttctttctttctggacttggatttagatctcagacagaagactagtgagcaccagagcatatcagtagtcctccgtggctcataatttatgatatacaccaatgcatgagtttctcaagtgtccaaaactcctctggaattattggagtatttgttaagcatatgagaaaaggatgtgttagtgaagggagagtgagaatggggagaacttggcgcgttctgtcttcccaagccttccctgcctcatctgaatacaccttctctattccaagaccatcagggtcgtgtgtccttgtgttgccttctcttgcaggttaccactgtgagccctacaacaaccctgcagacttcttccttgacgtcatcaacggagattcttcggctgtaatgttaaacagaggggaacaagaccatgaaggtatatgagtcttgtagattcatacgttggtgtccaattgtttgattgaagcccagtagtgtgtcttattaaagcatgcttttgtaggccagccaggactatgtagtgagatcctaccttaaaacaaaaatcgtgggttggggattttgctcagtggtagagcgcttgcctagaaagcgcaaggccctgggttcagtccccagctccgaaaaaaaagaaccaaa

SEQ ID NO: 3 - DNA, 28 bp, Rattus rattus
atctggaggaagactggagaacaagagc

SEQ ID NO: 4 - DNA, 28 bp, Rattus rattus
atattctggtaaggagccgggcaagagg

SEQ ID NO: 5 - DNA, 28 bp, Rattus rattus
atgacgtcaaggaagaagtctgcagggt

SEQ ID NO: 6 - DNA, 28 bp, Rattus rattus
acggagattcttcggctgtaatgttaaa

SEQ ID NO: 7 - DNA, 20 bp, Rattus rattus
ccccagtttgtggtctgcca

SEQ ID NO: 8 - DNA, 18 bp, Rattus rattus
gctaaaggtgaagatcta

SEQ ID NO: 9 - protein, 42 aa, Artificial Sequence
  SYNTHESIZED
QSGNLARQSGHLSRDRSALSRQSGNLARRSDALSRRSDALTQ

SEQ ID NO: 10 - protein, 42 aa, Artificial Sequence
  SYNTHESIZED
RSDHLSETSSDRTKRSDHLSAQSGSLTRRSDVLSEHSNARKT

SEQ ID NO: 11 - protein, 35 aa, Artificial Sequence
  SYNTHESIZED
QSGNLARQSGNLARRSDSLSTDNASRIRDRSNLTR

SEQ ID NO: 12 - protein, 35 aa, Artificial Sequence
  SYNTHESIZED
QSSDLSRRNDDRKKRREDLITTSSNLSRQSGHLSR